(19) United States
(12) Patent Application Publication
(10) Pub. No.: US 2025/0132039 A1
(43) Pub. Date: Apr. 24, 2025

(54) PREECLAMPSIA EVOLUTION PREDICTION, METHOD AND SYSTEM

(71) Applicant: NeoPrediX AG, Basel (CH)

(72) Inventors: Sven WELLMANN, Schierling (DE); Britta STEFFENS, Konstanz (DE); Gilbert KOCH, Überlingen (DE); Marvin HAEBERLE, Zuerich (CH); Marc PFISTER, Hilterfingen (CH)

(21) Appl. No.: 18/989,789

(22) Filed: Dec. 20, 2024

Related U.S. Application Data
(63) Continuation-in-part of application No. PCT/EP2023/066073, filed on Jun. 15, 2023.

(30) Foreign Application Priority Data
Jun. 21, 2022 (EP) 22180235.8

(51) Int. Cl. G16H 50/20; G16H 20/10

(52) U.S. Cl. CPC G16H 50/20; G16H 20/10

(57) ABSTRACT

A method for predicting health status of a subject, including: receiving at least one subject-related dynamic property data, receiving at least one subject-related covariate, processing the at least one subject-related dynamic property data and the at least one subject-related covariate data to generate a subject-related processed dataset, generating at least one health status hypothesis based on the subject-related processed dataset, and predicting at least one health status based on the at least one health status hypothesis. Also described is a system that can execute the method for predicting health status of a subject, including: at least one processing component; and at least one analyzing component, wherein the system is configured to predict at least one health status based on the at least one health status hypothesis.

### RELATED APPLICATIONS

This application is a bypass continuation-in-part of International Application No. PCT/EP2023/066073 filed on 15 Jun. 2023, which claims priority to European Application No. 22180235.8 filed 21 Jun. 2022, the entireties of which are incorporated herein by reference.

### FIELD

The invention lies in the field of prediction of a medical condition for a subject, and particularly in the field of predicting the evolution and/or development of preeclampsia in pregnant women and its effect on their children. The goal of the present invention is the provision of a method and system for predicting a potential medical outcome for pregnant women and/or their children. More particularly, the present invention relates to a system and a method performed in such a system and the corresponding use of the system. Preferably, the present invention relates to a system and a method performed in such a system, providing a treatment corresponding to the predicting of the evolution and/or development of preeclampsia.

### INTRODUCTION

Preeclampsia (PE) is a major cause of maternal and perinatal short and long-term morbidity and mortality worldwide (Tanner, 2022) affecting about 5% of all pregnancies (Mol, 2015). This fuels the need for better technologies and new approaches to lower the burden for people affected. PE is characterized by the new onset of hypertension in pregnancy or pre-existing hypertension when at least one new manifestation of end-organ dysfunction is present, which cannot be explained by a cause different than PE.

PE is one of the most severe pregnancy complications and poses a significant risk to both mothers and their children for morbidity and mortality. PE is a complex disease and diagnosis is challenging as pregnant women often have pre-existing morbidity overlapping with PE, e.g., pre-existing hypertension or compromised fetal growth. In black women, PE even affects up to 8% of pregnancies. Despite intensive investigation, it is still almost impossible to adequately predict, treat, or prevent PE.

Effects of PE on infant and mother extend many years after pregnancy, as evidenced by fetal programming of adult disease and increased risk of the development of maternal cardiovascular disease (Wellmann, 2014). Understanding PE's three key pathological stages in progression is crucial: (i) placental hypoxia and oxidative stress, (ii) excess release of anti-angiogenic and pro-inflammatory factors, and (iii) widespread systemic endothelial dysfunction and vasoconstriction (de Alwis, 2020).

During the past decades various guidelines on PE have evolved, representing state-of-the-art diagnostic approaches and best practices for early detection of the medical situation as reviewed recently by Scott et al. (Scott, 2022). In fact, the noble goal of all research and subsequent clinical care is to identify PE when mother and child are asymptomatic and to prevent them from reaching symptomatic stages.

MacDonald et al. presented a most recent review on clinical tools and biomarkers to predict PE (MacDonald, 2022). Peripheral blood biomarkers, namely soluble Fms-like Tyrosinkinase-1 (sFlt1) and placental growth factor (PIGF), show promising performance when used as a “rule-out” test, meaning to exclude subjects. However, the sensitivity to detect affected subjects is low. The review states several placental and cardiovascular biomarkers that could improve diagnostic performance in the future.

Jhee et al. (Jhee, 2019) presented a predictive model for late-onset PE, using different prediction models (e.g., logistic regression, decision tree, naïve Bayes classifier, support vector machine, etc.). The prediction of late-onset (i.e., after 34 weeks of gestation) was performed on a dataset using maternal characteristics and laboratory parameters at early second trimester. Detection rates of 77.1% were achieved, whereas the study endpoint was defined as new-onset hypertension accompanied with significant proteinuria.

Maric et al. (Maric, 2020) presented an approach focused on statistical analysis. A model is trained on all available clinical and laboratory data and enables to include a lot of missing values. Doppler imaging is neglected as feature, as this makes validation more difficult. This greatly increases applicability in different medical setups. Trained on the elastic net, this approach does not achieve performance measures that are high enough to be used in clinical settings.

However, these techniques do not present possibilities for continuous monitoring of ambulatory subjects, that is the continuous monitoring of patients that are allowed to move independently and maintain usual activities while seeking treatment and care. The subject's location should thus not be limited to a care facility and/or the techniques should not be limited to the presence of a medical professional.

### SUMMARY

In light of the above, it is therefore an object of the present invention to overcome or at least to alleviate the shortcomings and disadvantages of the prior art. More particularly, it is an object of the present invention to provide a method and a corresponding system for method for continuously predicting health status of an ambulatory subject with improved sensitivity and performance and less prone to yielding erroneous prediction of the at least one health status of the subject.

These objects are met by the present invention.

In a first aspect, the invention relates to a method for predicting health status of a subject, the method comprising: receiving at least one subject-related dynamic property data, receiving at least one subject-related covariate, processing the at least one subject-related dynamic property data and the at least one subject-related covariate data to generate a subject-related processed dataset, generating at least one health status hypothesis based on the subject-related processed dataset, and predicting at least one health status based on the at least one health status hypothesis. The method may also be a method for continuously predicting the health status of a subject and/or an ambulatory subject. The flexibility and/or modularity of the invention enables the invention to be present in proximity of the subject in a wide range of situations, such as but not limited to being away from medical facilities. This corresponds to a preferred advantage of the present invention. The method may also comprise individualizing the predicting according to at least one data related to the subject.

In one embodiment wherein step of predicting the at least one health status may be based on a computer-implemented dynamic model. The at least one health status hypothesis may comprise a correlation to at least one medical condition of the subject. It should be understood that the subject may be a female subject such as a pregnant woman and/or a non-pregnant woman. It should be understood that the term “female” as the subject is intended to refer to a female subject in reproductive age and/or childbearing age. Furthermore, the subject may also be a fetus and/or a neonate. In one embodiment, the method further may comprise predicting a dynamic behavior of the at least one health status of the subject. In another embodiment, the predicting of the at least one health status that may be based on a computer-implemented dynamic model, may be based on the generation of the at least one health status of the subject.

In a further embodiment, the method may comprise implementing at least one machine learning technique, wherein the method may comprise performing any of the preceding steps using the at least one machine learning technique.

The at least one subject-related covariate may comprise at least one biomarker. The at least one biomarker may be related to at least one medical condition, and the at least one biomarker may comprise at least one of: soluble Fms-like Tyrosinkinase-1 (sFlt1); placental growth factor (PIGF); neurofilament (NfL); C-terminal portion of arginine vasopressin (Copeptin); Albumin; Liver transaminase; Urea; Hemoglobin; Thrombocytes; Creatinine; Albuminuria; Proteinuria; estimated glomerular filtration rate (eGFR); creatinine clearance (CrCl); at least one additional kidney function measure; placental biomarkers such as placental RNAs, placental proteins; endothelial/cardiovascular biomarkers such as endothelial RNAs, endothelial proteins; or any combination thereof.

The at least one subject-related covariate may comprise at least one mother-related covariate, comprising at least one of: age; weight; height; body mass index (BMI); gravidity; parity; number of fetuses in a current pregnancy; ethnicity; body temperature; heart rate; heart rate variability; respiration rate; early membrane rupture; leukocytes; history of preeclampsia (family and mother); comorbidities such as gestational diabetes, obesity, cardiovascular/renal/kidney/thyroid diseases, autoimmune conditions, anemia, antiphospholipid syndrome, sexually transmitted diseases, headache; smoking habits before and/or during pregnancy; blood oxygen saturation (SpO2); blood pressure (systolic/diastolic); uteroplacental perfusion parameters; Doppler measurements of umbilical artery, middle cerebral artery, cerebroplacental ratio, uterine artery, fetal descending aorta, ductus venosus, umbilical vein, inferior vena cava, pulsatility index in uterine arteries; soft-tissue parameters such as fractional arm volume and fractional thigh volume; and at least one measurement of the at least one biomarker.

The at least one subject-related covariate may comprise at least one fetus-related covariate comprising at least one of: gender; fetal weight during pregnancy; fetal biometric parameters such as femur length, abdominal circumference, head circumference, mid-thigh circumference and biparietal diameter; rates of small for gestational age; gestational age; heart rate, heart rate variability; respiration rate; uteroplacental perfusion parameters; and at least one measurement of the at least one biomarker. Additionally or alternatively, the at least one subject-related covariate may comprise at least one neonate-related covariate, comprising at least one of: gender; birth weight; body weight; body length; gestational age at birth; postnatal age; temperature; heart rate; heart rate variability; respiration rate; breastfeeding duration; exclusive breastfeeding duration; pH value; breath aid; oxygen demand; blood oxygen saturation (SpO2); blood pressure (systolic/diastolic); Apgar scores; at least one additional neonatal biometric parameter; and at least one measurement of the at least one biomarker. Additionally or alternatively, the at least one subject-related covariate may comprise at least one environmental covariate comprising at least one of: country of residence, country of birth, day and time of birth, humidity conditions at birth, and surrounding temperature at birth.

Furthermore, the method may comprise synchronizing the at least one subject-related covariate and/or the at least one subject-related property data with respect to time. The method may comprise synchronizing the at least one subject-related covariate and/or the at least one subject-related property data according to at least one timestamp comprised in the at least one subject-related covariate and/or at least one subject-related property data. The at least one timestamp may represent the date and/or time the at least one subject-related covariate and/or the at least one subject-related property data was recorded. The at least one timestamp may be recorded with a resolution in the range of seconds, minutes, hours, days, weeks and/or months. The method may comprise modifying the resolution of the timestamp of the at least one subject-related covariate and/or the at least one subject-related property data. The method may comprise modifying the resolution of the timestamp of the at least one subject-related covariate and/or the at least one subject-related property data, into a standardized timestamp resolution. The method may comprise modifying the impact of the at least one subject-related covariate and/or the at least one subject-related property data, on at least one output of the method, according to the timestamp and/or according to the at least one subject-related covariate and/or the at least one subject-related property data. This step would allow data to impact the outputs of the method according to how recent the data recorded, according to the type of data recorded and according to both the time and type of data recorded, as some data samples coming from an ultrasound scan may have a different impact according to the time it was recorded at compared to blood pressure (systolic/diastolic) recorded at another time. This presents a further advantage of the present invention.

Moreover, the method may comprise synchronizing the at least one subject-related covariate and/or the at least one subject-related property data with respect to each other. The method may comprise synchronizing the at least one subject-related covariate and/or the at least one subject-related property data relatively to at least one data, wherein the at least one data may comprise at least one subject-related covariate and/or at least one subject-related property data.

Additionally and/or alternatively, the method may comprise converting the data type of the at least one subject-related covariate and/or the at least one subject-related property data, into a standardized data type. The method may comprise removing at least one outlier comprised in the at least one subject-related covariate and/or the at least one subject-related property data.

In one embodiment, the method may comprise generating at least one threshold, wherein the at least one threshold expresses an indication of at least one potential medical condition. In a further embodiment, the method may comprise outputting at least one potential medical condition, wherein the at least one potential medical condition may comprise at least one of: seizures; respiratory; cardiovascular; hematological dysfunction; endocrine; renal; hepatic; uteroplacental dysfunction; fetal growth restriction; unplanned preterm birth; placental abruption; hemolysis elevated liver enzymes, low platelets (HELLP) syndrome; and eclampsia. It should be understood that PE may cause multiple organ involvement, namely central nervous system (CNS) including seizures, respiratory, cardiovascular, hematological dysfunction, endocrine, renal, hepatic, and uteroplacental dysfunction. Moreover, complications of PE may include but are not limited to: fetal growth restriction as PE affects the arteries carrying blood to the placenta; preterm birth. Furthermore, PE may lead to an unplanned preterm birth, i.e., delivery before 37 weeks. It should, therefore, be noted that this invention is applicable to gestational hypertension and gestational diabetes, and is associated with maternal, fetal and neonatal complications of these medical conditions in pregnant women.

In a further embodiment, the method may comprise of determining a minimum threshold value for the at least one threshold, and determining a maximum threshold value for the at least one threshold. Moreover, the at least one subject-related data may be under the minimum threshold value, the method may comprise outputting a monitoring suggestion. When the at least one subject-related data may be above the maximum threshold value, the method may comprise outputting a treatment suggestion. The method may also comprise outputting a treatment according to the predicting of the health status of the subject. Further, the method may comprise determining a baseline for the at least one subject-related data. Additionally, or alternatively, the method may comprise determining at least one intermediate threshold value, wherein the at least one intermediate threshold may comprise at least one value between the minimum threshold value and the maximum threshold value.

In one embodiment, the method may comprise: correlating at least a range of each of the at least one intermediate to the at least one medical condition, generating an interpreted dataset based on the correlation step, and outputting an automated report indicating the at least one potential medical condition. In a further embodiment, the method may comprise: determining at least one medical condition change indicator, monitoring changes of the at least one medical condition change indicator, generating at least one medical condition change indicator trend, and predicting an evolution of at least one of the at least one medical condition, wherein the prediction may be based on the at least one medical condition change indicator trend. Moreover, the method may comprise monitoring at least one value change of the at least one subject-related property, the method comprising: recording an initial value of the at least one subject-related property, recording at least one subsequent value of the at least one subject-related property, contrasting the initial value with at least one of the at least one subsequent value, generating a compared value data, and outputting a subject-related property hypothesis based on the compared value data. The step of recording at least one subsequent value may comprise recording one current value of the at least one subject-related property, wherein the current value may be different from the initial value. The method thus may provide an unbiased support decision to a medical professional, wherein the unbiased support decision may be tailored to the subject. This presents a further advantage of the present invention.

In one embodiment, the method may be a non-diagnostic method. In another embodiment, the method may be a diagnostic method.

The method may also comprise carrying out the method steps as recited herein using data from at least one database. The at least one database may comprise at least one of: public health database, subject's individual database, medical professional's database, healthcare provider's database, and private databanks. Additionally, or alternatively, the method may comprise: feeding data to the at least one server, training the computer-implemented dynamic model based on data fed to the at least one server, and generating an adjusting function based on the training data, wherein the adjusting function may be suitable for adjusting any steps of the method according to any of the preceding method embodiments.

In one embodiment, the method may comprise triggering at least one action suggestion based on the at least one health status hypothesis. Additionally, or alternatively, the method may comprise displaying the at least one action suggestion to a user. In another embodiment, method may comprise prompting the user to input at least one of: acceptation of at least one of the at least one action suggestion, and rejection of at least one of the at least one action suggestion. The user may reject at least one of the at least one action suggestion, the method may comprise prompting the user to provide at least one annotation. The method may comprise utilizing at least one acceptation and/or the at least one rejection for optimizing at least one algorithm implemented by the method. The method may comprise utilizing at least one acceptation and/or the at least one rejection for reinforcement learning. The method may comprise utilizing at least one acceptation and/or the at least one rejection for online learning. Making use of the feedback related to the at least one action suggestion allows for continuous learning of the algorithms, even while in use. This constitutes an advantage of the present invention. Making use of the feedback related to the at least one action suggestion specific to the subject allows for continuous learning of the algorithms, tailored to the subject. This constitutes a further advantage of the present invention. The computer-implemented dynamic model may be based on: a Bayesian statistical approach; an artificial neural network (ANN) approach; a convolutional neural network (CNN) approach; a recurrent neural network (RNN) approach; a pharmacometrics (PMX) modeling and/or simulation approach; a supervised learning approach; a reinforcement learning approach, a deep learning (DL) approach, a multi-layer neural network approach, a hybrid approach and/or an explainable AI (XAI) concept. The computer-implemented dynamic model may also be based on decision tree approaches and/or random forests approaches. Decision tree algorithms may comprise splitting at least one dataset into subsets based on feature values, aiming to create branches that lead to increasingly pure or homogeneous subsets with respect to the target variable. This splitting process may continue recursively, creating a tree structure where each node represents a decision based on a feature, and each leaf node represents a predicted outcome. Random forests may enhance decision trees by building an ensemble of trees, each trained on a random subset of the data and a random selection of features. The predictions from all trees are combined (e.g., by majority vote for classification and/or or averaging for regression), which increases accuracy and reduces the risk of overfitting, making random forests more robust than individual decision trees.

The at least one medical condition may comprise at least one of: fetal growth-related condition; PE-related condition; gestational diabetes-related condition; gestational hypertension-related condition; gestational medical treatment such as cyclooxygenase inhibitors, e.g., Aspirin; at least one relevant medication; and at least one potential medical condition.

Moreover, the method may comprise correlating the at least one biomarker to at least one medical condition, wherein the at least one medical condition may comprise a potential disease. The method may also comprise predicting occurrence of the at least one hypothesis at a given time period, wherein the method further may comprise recognizing a plurality of different time periods comprising at least one of: prenatal period; pregnancy; delivery period and postnatal period. In one embodiment, the method may comprise outputting a likelihood of occurrence correlated to each of the time periods. Further, the method may comprise executing at least one machine learning (ML) algorithm. The at least one ML algorithm may comprise a supervised algorithm architecture, an unsupervised algorithm architecture, or any combination thereof. Additionally or alternatively, the at least one ML algorithm may comprise at least one artificial deep learning (DL) architecture, wherein the at least one artificial DL architecture may comprise at least one of: ANN, CNN, and RNN. The unsupervised algorithm architecture may comprise implementing at least one clustering approach of at least one cluster. Furthermore, the at least one analytical approach may comprise at least one of pattern recognition, probabilistic modeling, Bayesian schemes, reinforcement learning, statistical analytics, statistical models, principal component analysis (PCA), independent component analysis, dynamic time warping, maximum likelihood estimates (MLE), modeling, estimating, neural network (NN), CNN, RNN, deep convolutional network, DL, ultra-deep learning, genetic algorithms, Markov models, and/or hidden Markov models.

In a further embodiment, the method may comprise implementing at least one pharmacometrics (PMX) model. The at least one PMX model may comprise a computer-implemented PMX model comprising at least one of: mathematical-statistical pharmacokinetics-pharmacodynamics (PK-PD) model; physiology-based PK (PBPK) model; physiology-based PK-PD (PBPKPD) model; drug exposure-efficacy response model; and drug exposure-safety response model. The at least one PMX model may quantify the relationships between drug dosing, drug concentration in the subject's body, and the resulting effects on the subject. The at least one PMX model may allow the customization of the drug dosing, drug concentration according to at least one subject-related biomarker, covariate and/or medical condition, and/or previous subject-related biomarker, covariate and/or medical condition while preserving the anonymity of the at least one previous subject. This presents a preferred advantage of the present invention.

The at least one health status of the subject may comprise: PE, gestational diabetes, fetal growth-related conditions and/or gestational hypertension-related conditions. In one embodiment, the step of predicting the at least one health status based on the at least one health status hypothesis, may comprise using at least one fetal growth-related data. In another embodiment, the step of predicting the at least one health status may comprise using at least one fetal growth-related data, wherein the at least one health status hypothesis may be based on the at least one fetal growth-related data. The at least one fetal growth-related data may be retrieved from at least one of the at least one database. In a further embodiment, the step of predicting the at least one health status may comprise using at least one PE-related data, wherein the at least one health status hypothesis may be based on at least one PE-related data. The at least one PE-related data may be retrieved from at least one of the at least one database.

Furthermore, the method may comprise determining at least one drug based on the at least one health status, wherein the at least one drug may be suitable for preventing occurrence and/or recurrence of the at least one medical condition and/or the at least one health status. The method may comprise determining at least one drug based on the at least one health status, wherein the at least one drug may be suitable for treating the at least one medical condition and/or the at least one health status. The at least one drug comprising at least one of: Aspirin; Ibuprofen; at least one corticosteroid drug; at least one anti-hypertensive drug; and at least one cardiovascular-related drug. Moreover, the method may comprise generating at least one drug administration route, wherein the at least one drug administration route may comprise at least one of: intravenous; intramuscular; subcutaneous; inhalation; transdermal; transcutaneous; oral; rectal; and sublingual. The method may comprise generating at least one dosing regimen of the at least one drug. Additionally or alternatively, the method further may comprise optimizing the at least one dosing regimen, wherein the at least one dosing regimen may comprise at least one of: the at least one drug; the at least one drug administration route; at least one dose scheme; at least one drug administration duration; and at least one drug administration frequency. The step of optimizing the at least one dosing regimen may be based on the at least one health status hypothesis. The method may also comprise implementing at least one optimal control theory, wherein the at least one optimal control theory is computer-implemented. It should be understood that the method as recited herein is a computer-implemented method. The method may comprise optimizing at least one ongoing treatment of the at least one medical condition. The method may comprise optimizing at least one ongoing treatment of the at least one potential medical condition. The step of optimizing may be based on the at least one health status hypothesis and/or the at least one health status. The method may comprise generating at least one treatment suggestion, wherein the at least one treatment suggestion may be based on the at least one health status hypothesis and/or the at least one health status. The method may comprise optimizing the at least one treatment suggestion, wherein the method may comprise carrying out the optimizing step after executing the at least one treatment suggestion.

In one embodiment, the method may comprise: adapting any of the preceding method embodiments to the subject, and generating at least one individualized treatment protocol, wherein the at least one individualized treatment protocol may be based on the at least one health status of the subject. The method may comprise optimizing the at least one individualized treatment protocol, wherein the method may comprise carrying out the optimizing step after executing the at least one individualized treatment protocol.

Furthermore, the method may comprise implanting any of the preceding optimizing steps assisted by a computer-implemented pharmacometrics approach. The method may comprise carrying out the method as recited herein in absence of the subject. Furthermore, the method may comprise carrying out the method as recited herein using at least one historical data. The at least one historical data may be a historical data of the subject. The at least one historical data may comprise data from at least one of: public health database, subject's individual database, medical professional's database, healthcare provider's database, and private databanks.

In another embodiment, the method may comprise at least one of: capturing at least one image data of the subject; and receiving at least one image data of the subject, wherein the at least one image data may comprise data relevant to at least one medical condition and/or at least one potential medical condition of the subject. Moreover, the method is suitable for implementation in at least one medical device such as an ultrasound device.

In a second aspect, the invention relates to a system for predicting health status of a subject, the system comprising: at least one processing component configured to receive at least one subject-related dynamic property data, receive at least one subject-related covariate, and process the at least one subject-related dynamic property data and the at least one subject-related covariate data to generate a subject-related processed dataset; at least one analyzing component configured to analyze the subject-related processed dataset, and generate at least one health status hypothesis based on the subject-related processed dataset, wherein the system is configured to predict at least one health status based on the at least one health status hypothesis. The system may also be a system for continuously predicting the health status of a subject and/or an ambulatory subject. The flexibility and/or modularity of the invention enables the invention to be present in proximity of the subject in a wide range of situations, such as but not limited to being away from medical facilities. This corresponds to a preferred advantage of the present invention. The system may also be configured to individualize the predicting according to at least one data related to the subject.

Furthermore, the system may comprise at least one storing component configured to store data relevant to the at least one health status of the subject. The system may also comprise at least one computing component configured to implement dynamic model for predicting the at least one health status. The at least one health status hypothesis may comprise a correlation to at least one medical condition of the subject.

The subject may be: a neonate, a fetus, and/or a female, wherein the female may be at least one of: a pregnant woman, and a non-pregnant woman.

The system may be configured to predict a dynamic behavior of the at least one health status of the subject. The system may be configured to perform any of the steps according to any of the preceding method embodiments by means of the at least one machine learning technique.

The at least one subject-related covariate may comprise at least one biomarker. The at least one biomarker may be related to at least one medical condition, and the at least one biomarker may comprise at least one of: soluble Fms-like Tyrosinkinase-1 (sFlt1); placental growth factor (PIGF); neurofilament (NfL); C-terminal portion of arginine vasopressin (Copeptin); Albumin; Liver transaminase; Urea; Hemoglobin; Thrombocytes; Creatinine; Albuminuria; Proteinuria; estimated glomerular filtration rate (eGFR); creatinine clearance (CrCl); at least one additional kidney function measure; placental biomarkers such as placental RNAs, placental proteins; endothelial/cardiovascular biomarkers such as endothelial RNAs, endothelial proteins; or any combination thereof.

The at least one subject-related covariate may comprise at least one neonate-related covariate comprising at least one of: gender; race; birth weight; gestational age; birth mode such as vaginal, vacuum extraction, cesarean; temperature; heart rate; respiration rate; pH value; umbilical cord pH value; breath aid; oxygen demand; blood oxygen saturation (SpO2); blood pressure (systolic/diastolic); Apgar scores; and at least one measurement of the at least one biomarker. Moreover, the at least one subject-related covariate may comprise at least one mother-related covariate comprising at least one of: age; race; early membrane rupture; temperature; risk factors such as diabetes, adiposity, gravidity, parity, leukocytes; and at least one measurement of the at least one biomarker. Additionally or alternatively, the at least one subject-related covariate may comprise at least one fetus-related covariate comprising at least one of: gender; fetal weight during pregnancy; fetal biometric parameters such as femur length, abdominal circumference, head circumference, mid-thigh circumference and biparietal diameter; rates of small for gestational age; gestational age; heart rate, heart rate variability; respiration rate; uteroplacental perfusion parameters; and at least one measurement of the at least one biomarker. Further, the at least one subject-related covariate may comprise at least one environmental covariate comprising at least one of: country of residence; country of birth; day and time of birth; humidity conditions at birth; and surrounding temperature at birth.

Furthermore, the system may be configured to synchronize the at least one subject-related covariate and/or the at least one subject-related property data with respect to time. The system may be configured to synchronize the at least one subject-related covariate and/or the at least one subject-related property data according to at least one timestamp comprised in the at least one subject-related covariate and/or at least one subject-related property data. The at least one timestamp may represent the date and/or time the at least one subject-related covariate and/or the at least one subject-related property data was recorded. The at least one timestamp may be recorded with a resolution in the range of seconds, minutes, hours, days, weeks and/or months. The system may be configured to modify the resolution of the timestamp of the at least one subject-related covariate and/or the at least one subject-related property data. The system may be configured to modify the resolution of the timestamp of the at least one subject-related covariate and/or the at least one subject-related property data, into a standardized timestamp resolution. The system may be configured to modify the impact of the at least one subject-related covariate and/or the at least one subject-related property data, on at least one output of the system, according to the timestamp and/or according to the at least one subject-related covariate and/or the at least one subject-related property data. The system would thus allow data to impact the outputs of the system according to how recent the data recorded, according to the type of data recorded and according to both the time and type of data recorded, as some data samples coming from an ultrasound scan may have a different impact according to the time it was recorded at compared to blood pressure (systolic/diastolic) recorded at another time. This presents a further advantage of the present invention.

Moreover, the system may be configured to synchronize the at least one subject-related covariate and/or the at least one subject-related property data with respect to each other. The system may be configured to synchronize the at least one subject-related covariate and/or the at least one subject-related property data relatively to at least one data, wherein the at least one data may be configured to at least one subject-related covariate and/or at least one subject-related property data.

Additionally and/or alternatively, the system may be configured to convert the data type of the at least one subject-related covariate and/or the at least one subject-related property data, into a standardized data type. The system may be configured to remove at least one outlier comprised in the at least one subject-related covariate and/or the at least one subject-related property data.

The system may be configured to generate at least one threshold, wherein the at least one threshold expresses an indication of at least one potential medical condition. The system may be configured to output at least one potential medical condition, wherein the at least one potential medical condition may comprise at least one of: seizures; respiratory; cardiovascular; hematological dysfunction; endocrine; renal; hepatic; uteroplacental dysfunction; fetal growth restriction; unplanned preterm birth; placental abruption; hemolysis elevated liver enzymes, low platelets (HELLP) syndrome; and eclampsia. The at least one analyzing component may be configured to: determine a minimum threshold value for the at least one threshold and determine a maximum threshold value for the at least one threshold. When the at least one subject-related data may be under the minimum threshold value the at least one analyzing component outputs a monitoring suggestion. When the at least one subject-related data may be above the maximum threshold value the at least one analyzing component outputs a treatment suggestion. The at least one analyzing component may also be configured to output a treatment according to the predicting of the health status of the subject. The at least one analyzing component may be configured to determining a baseline for the at least one subject-related data. The at least one analyzing component may be configured to determine at least one intermediate threshold value, wherein the at least one intermediate threshold value may comprise at least one value between the minimum threshold value and the maximum threshold value. The at least one analyzing component may be configured to: correlate at least a range of each of the least one intermediate threshold value correlated to at least one medical condition, generate an interpreted dataset based on the correlation step, and output an automated report indicating at least one potential medical condition. The at least one analyzing component may be configured to: determine at least one medical condition change indicator, monitor changes of the at least one medical condition change indicator, generate at least one medical condition change indicator trend, and predict an evolution of at least one of the at least one medical condition based on the at least one medical condition change indicator trend.

In a further embodiment, the system may comprise at least one monitoring component configured to monitor at least one value change of the at least one subject-related property, wherein the at least one monitoring component may be further configured to: record an initial value of the at least one subject-related property, record at least one subsequent value of the at least one subject-related property, contrast the initial value with at least one of the at least one subsequent value, generate a compared value data, and output a subject-related property hypothesis based on the compared value data. The at least one monitoring component may be configured to record one current value of the at least one subject-related property, wherein the current value may be different from the initial value. The system thus may provide an unbiased support decision to a medical professional, wherein the unbiased support decision may be tailored to the subject. This presents a further advantage of the present invention.

The system may be a non-diagnostic system. In another embodiment, the system may be a diagnostic system.

The system may be configured to carry out the method steps according to any of the preceding method embodiments using data from at least one database. The at least one database may comprise at least one of: public health database, subject's individual database, medical professional's database, healthcare provider's database, and private databanks. Furthermore, the system may be configured to: feed data to the at least one server, train the computer-implemented dynamic model based on data fed to the at least one server, and generate an adjusting function based on the training data, wherein the adjusting function may be suitable for adjusting any configuration of the system according to any of the preceding system embodiments. The system may be configured to trigger at least one action suggestion based on the at least one health status hypothesis and/or the at least one health status. The system may be configured to display the at least one action suggestion to a user.

In one embodiment, the system may be configured to prompt the user to input at least one of: acceptation of at least one of the at least one action suggestion, and rejection of at least one of the at least one action suggestion. When the user rejects at least one of the at least one action suggestion, the system may be configured to prompt the user to provide at least one annotation. The system may be configured to utilize at least one acceptation and/or the at least one rejection for optimizing at least one algorithm implemented by the system. The system may be configured to utilize at least one acceptation and/or the at least one rejection for reinforcement learning. The system may be configured to utilize at least one acceptation and/or the at least one rejection for online learning. Making use of the feedback related to the at least one action suggestion allows for continuous learning of the algorithms, even while in use. This constitutes an advantage of the present invention. Making use of the feedback related to the at least one action suggestion specific to the subject allows for continuous learning of the algorithms, tailored to the subject. This constitutes a further advantage of the present invention. The computer-implemented dynamic model may be based on: a Bayesian statistical approach, an artificial neural network (ANN) approach, a convolutional neural network (CNN) approach, a recurrent neural network (RNN) approach, a pharmacometrics (PMX) approach, a supervised learning approach, a reinforcement learning approach, a deep learning (DL) approach, a multi-layer neural network approach, a hybrid approach and/or an explainable AI (XAI) concept. The computer-implemented dynamic model may also be based on decision tree approaches and/or random forests approaches. Decision tree algorithms may comprise splitting at least one dataset into subsets based on feature values, aiming to create branches that lead to increasingly pure or homogeneous subsets with respect to the target variable. This splitting process may continue recursively, creating a tree structure where each node represents a decision based on a feature, and each leaf node represents a predicted outcome. Random forests may enhance decision trees by building an ensemble of trees, each trained on a random subset of the data and a random selection of features. The predictions from all trees are combined (e.g., by majority vote for classification and/or or averaging for regression), which increases accuracy and reduces the risk of overfitting, making random forests more robust than individual decision trees.

The at least one medical condition may comprise at least one of: fetal growth-related condition; neonatal thyroid dysfunction; PE-related condition; gestational diabetes related condition; gestational hypertension-related condition; and gestational thyroid dysfunction. The system may be configured to correlate the at least one biomarker to at least one medical condition, wherein the at least one medical condition may comprise a potential disease. The system may also be configured to predict occurrence of the at least one hypothesis at a given time period, wherein the system may be configured to recognize a plurality of different time periods comprising at least one of: prenatal period, pregnancy, delivery period, and postnatal period. The system may be configured to output a likelihood of occurrence correlated to each of the time periods. Further, the system may be configured to execute at least one machine learning (ML) algorithm, wherein the at least one ML algorithm may comprise a supervised algorithm architecture, an unsupervised algorithm architecture, or any combination thereof. The at least one ML algorithm may comprise at least one artificial deep learning (DL) architecture, wherein the at least one artificial DL architecture may comprise at least one of: ANN, CNN, and RNN. The unsupervised algorithm architecture may be configured to implement at least one clustering approach of at least one cluster.

Moreover, the system may be configured to execute at least one analytical approach, wherein the at least one analytical approach may comprise at least one of pattern recognition, probabilistic modeling, Bayesian schemes, reinforcement learning, statistical analytics, statistical models, principal component analysis (PCA), independent component analysis, dynamic time warping, maximum likelihood estimates (MLE), modeling, estimating, neural network (NN), convolutional neural network (CNN), recurrent neural network (RNN), deep convolutional network, deep learning (DL), ultra-deep learning, genetic algorithms, Markov models, and/or hidden Markov models. The system may be configured to implement at least one pharmacometrics (PMX) model, the at least one PMX model may comprise at least one of: mathematical-statistical pharmacokinetic-pharmacodynamic (PK-PD) model; physiology-based PK (PBPK) model; physiology-based PK-PD (PBPKPD) model; drug exposure-efficacy response model; and drug exposure-safety response model. The at least one PMX model may quantify the relationships between drug dosing, drug concentration in the subject's body, and the resulting effects on the subject. The at least one PMX model may allow the customization of the drug dosing, drug concentration according to at least one subject-related biomarker, covariate and/or medical condition, and/or previous subject-related biomarker, covariate and/or medical condition while preserving the anonymity of the at least one previous subject. This presents a preferred advantage of the present invention.

The system may be configured to predict the at least one health status based on the at least one health status hypothesis and use at least one fetal growth-related data. The system may be configured to predict the at least one health status using at least one fetal growth-related data, wherein the at least one health status hypothesis may be based on the at least one fetal growth-related data. The at least one fetal growth-related data may be retrieved from at least one of the at least one database. The system may be configured to predict the at least one health status, may comprise using at least one PE-related data, wherein the at least one health status hypothesis may be based at least one PE-related data. The at least one PE-related data may be retrieved from at least one of the at least one database.

The system may comprise at least one imaging component configured to at least one of: capture at least one image data of the subject and receive at least one image data of the subject, wherein the at least one image data may comprise data relevant to at least one medical condition and/or at least one potential medical condition of the subject.

Moreover, the system is further configured to perform any of the method steps as recited herein.

The system may comprise at least one implementation component configured to connect the system to at least one medical device such as an ultrasound device, wherein the system once connected to the at least one medical device is configured to perform any of the steps according to the method as recited herein. The system may be configured to operate in absence of the subject.

Furthermore, the method comprises using the system as recited herein to perform any of the steps of the method as recited herein.

In a third aspect, the invention relates to a method of treatment for treating a medical condition of a subject, wherein the treatment comprises generating a treatment protocol comprising at least one treatment drug and a treatment regimen, wherein the treatment regimen is based on at least one health status hypothesis. The at least one health status hypothesis may be provided by the method as recited herein. The at least one drug may be provided by the method as recited herein. The at least one health status of the subject may comprise: PE, gestational diabetes, and/or fetal growth issues.

The treatment further may comprise treating the subject for at least one potential medical condition previous to onsetting of the at least one medical condition. The subject may be at least one of: a pregnant woman, and a non-pregnant woman, a fetus, and/or a neonate.

In a fourth aspect, the invention relates to a diagnostic method for diagnosing a medical condition of a subject, wherein the diagnosis comprises generating at least one diagnostic finding comprising at least one medical condition of the subject, wherein the at least one diagnostic finding is based on at least one health status hypothesis. The diagnostic method may comprise generating at least one method of treatment, wherein the at least one method of treatment may be for treating the at least one medical condition of the subject. The diagnostic method may comprise generating at least one treatment according to the diagnosis of the medical condition of the subject. For example, after the method may comprise outputting an operational command to a drug dispenser, wherein the operational command may comprise outputting at least one drug corresponding to the treatment generated by the method according to the predicting step of the method. The diagnostic method may comprise generating the at least one diagnostic finding, wherein the at least one diagnostic finding may comprise the at least one medical condition of the subject previous to onset of the at least one medical condition. The diagnostic method comprising at least one preventive method of treatment, wherein the at least one preventive method of treatment may be for treating the at least one medical condition of the subject previous to onset of the at least one medical condition. The at least one health status hypothesis may be provided by the method according to any of the preceding method embodiments.

The diagnostic method may comprise providing at least one drug, wherein the at least one drug may be provided by the method according to any of the preceding method embodiments. The at least one health status of the subject may comprise: PE, gestational hypertension, gestational diabetes and/or fetal growth issues. The subject may be at least one of: a pregnant woman, a non-pregnant woman, a fetus, and/or a neonate. The diagnostic method may comprise suggesting a method of treating according to any of the preceding method of treatment embodiments.

In a fifth aspect, the invention relates to use of the system as recited herein for carrying out the method as recited herein. The method may comprise prompting the system as recited herein to perform the steps of the method as recited herein. Use of the method as recited herein for implementing the method of treatment as recited herein. Use of the method as recited herein for implementing the diagnostic method as recited herein. Use of the method as recited herein for implementing the diagnostic method as recited herein and the method of treatment as recited herein, wherein implementing the diagnostic method precedes implementing the method of treatment.

In simple words, the presented invention relates to predicting diseases in the field of perinatal medicine. More specifically, the approach of the present invention enables combining different components such as machine learning, data augmentation, artificial intelligence, dynamical pharmacometrics and pharmaceutics suitable for neonatology and obstetrics. Moreover, the present invention enables using a plurality of PE-related biomarkers to detect and monitor maternal, fetal and neonatal stress factors in clinical studies over time, e.g., during the last 15 years. As PE is a progressive multi-system disease, the present invention allows to test multiple PE-related biomarkers such as (i) cardiovascular markers at triage (Wellmann 2014), (ii) biomarkers to detect and monitor subclinical maternal end-organ dysfunction such as Copeptin for renal system (Wellmann 2014) and NfL for central nervous system (Evers 2018), and (iii) biomarkers for diagnosis and monitoring of fetal stress reaction (Burkhardt 2012) and adverse neonatal outcome (Letzner 2011), (Depoorter 2018). This is particularly advantageous, as a combined analysis of such biomarkers allows the present invention to predict the health status of the subject. It should be understood that prediction of health status may comprise a current, future and/or past health status of the subject. That is, the present invention may allow to predict a future health status of the subject previous to onset of a medical condition, and may also allow to predict a current health status of the subject previous to onset of a medical condition. Altogether, the invention provides an integrated approach, which comprises combining and utilizing multi-dimensional and longitudinal data, processing of data assisted by a computer-implemented method, leveraging AI-and PMX-based computer-implemented models to personalize and optimize prevention, diagnosis, management, and treatment of PE. The present invention is also advantageous as it allows to avoid PE-related complications in a subject or group of subjects, such as complications in mothers and their unborn and born children. Thus, the present invention combines available multi-source input to improve perinatal prevention, diagnosis and management of PE and its complications, wherein the method of the present invention allows such a combination to be carried out without need for an intervention of a human, as the computer-implemented method allows utilizing sequential multi-source data to achieve: integration of data at all levels by optimizing prevention, diagnosis and management of the disease, providing a solution that is able to alleviate prenatal (i.e., mother's and fetus'), but also postnatal (i.e., mother's and neonate's) morbidity, utilizing intelligent integration concepts of multiple components, including clinical data, biomarkers, uteroplacental perfusion and fetal-growth data, signal-processing data, together with longitudinal measurements, combine ML and other AI methods with pharmacological principles and innovative dynamic pharmacometrics computer models, and leverage pharmacometrics computer-implemented simulation approaches to optimize and personalize dosing to maximize efficacy/safety balance not just for mothers but also for their unborn and born children. This approach is particularly advantageous as it renders a more precise, effective and efficient method and a corresponding system or method for predicting health status of the subject with improved sensitivity and performance and less prone to yielding erroneous prediction of the at least one health status of the subject.

The system may comprise at least one implementation component configured to connect the system to at least one medical device such as a sensor, wherein the system once connected to the at least one medical device may be configured to perform any of the steps according to any of the preceding method embodiments. The system may comprise at least one implementation component configured to connect the system to at least one medical device such as but not limited to a drug dispenser, wherein the system once connected to the at least one medical device may be configured to perform any of the steps according to any of the preceding method embodiments. The components of the system may be configured to transmit and/or receive data to and/or from each other regardless of the distance between each component. The components may be connected to a server and the users, comprising but not limited to a medical professional and/or the subject, may access at least one data from the server from a user terminal.

The present technology is also described by the following numbered embodiments.

Below, method embodiments will be discussed. These embodiments are abbreviated by the letter “M” followed by a number. When reference is herein made to a method embodiment, those embodiments are meant.
M1. A method for predicting health status of a subject, the method comprising
   receiving at least one subject-related dynamic property data,
   receiving at least one subject-related covariate,
   processing the at least one subject-related dynamic property data and the at least one subject-related covariate data to generate a subject-related processed dataset,
   generating at least one health status hypothesis based on the subject-related processed dataset, and
   predicting at least one health status based on the at least one health status hypothesis.
M2. The method according to the preceding embodiment, wherein step of predicting the at least one health status is based on a computer-implemented dynamic model.
M3. The method according to any of the preceding method embodiments, wherein the at least one health status hypothesis comprises a correlation to at least one medical condition of the subject.
M4. The method according to any of the preceding method embodiments, wherein the subject is a female subject.
M5. The method according to the preceding embodiment, wherein the female subject is a pregnant woman.
M6. The method according to any of the 2 preceding embodiments, wherein the female subject comprises a non-pregnant woman.
M7. The method according to any of the preceding method embodiments, wherein the subject is a fetus.
M8. The method according to any of the preceding method embodiments, wherein the subject is a neonate.
M9. The method according to any of the preceding method embodiments, wherein the method further comprises predicting a dynamic behavior of the at least one health status of the subject.
M10. The method according to any of the preceding method embodiments, wherein the method comprises implementing at least one machine learning technique, wherein the method comprises performing any of the preceding steps using the at least one machine learning technique.
M11. The method according to any of the preceding method embodiments, wherein the at least one subject-related covariate comprises at least one biomarker.
M12. The method according to the preceding embodiment, wherein the at least one biomarker is related to at least one medical condition.
M13. The method according to any of the 3 preceding embodiments, wherein the at least one biomarker comprises at least one of: soluble Fms-like Tyrosinkinase-1 (sFlt1); placental growth factor (PIGF); neurofilament (NfL); C-terminal portion of arginine vasopressin (Copeptin); Albumin; Liver transaminase; Urea; Hemoglobin; Thrombocytes; Creatinine; Albuminuria; Proteinuria; estimated glomerular filtration rate (eGFR); creatinine clearance (CrCl); at least one additional kidney function measure; placental biomarkers such as placental RNAs, placental proteins; endothelial/cardiovascular biomarkers such as endothelial RNAs, endothelial proteins; or any combination thereof.
M14. The method according to any of the preceding method embodiments and with features of embodiments M4 to M6, wherein the at least one subject-related covariate comprises at least one mother-related covariate, comprising at least one of: age; weight; height; body mass index (BMI); gravidity; parity; number of fetuses in a current pregnancy; ethnicity; body temperature; heart rate; heart rate variability; respiration rate; early membrane rupture; leukocytes; history of PE (family and mother), comorbidities such as gestational diabetes, obesity, cardiovascular/renal/kidney/thyroid diseases, autoimmune conditions, anemia, antiphospholipid syndrome, sexually transmitted diseases, headache; smoking habits s before and/or during pregnancy; blood oxygen saturation (SpO2); blood pressure (systolic/diastolic); utero-placental perfusion parameters; doppler measurements of: umbilical artery, middle cerebral artery, cerebroplacental ratio, uterine artery, fetal descending aorta, ductus venosus, umbilical vein, inferior vena cava, pulsatility index in uterine arteries; soft-tissue parameters such as fractional arm volume and fractional thigh volume; and at least one measurement of the at least one biomarker.
M15. The method according to any of the preceding method embodiments and with features of embodiment M7, wherein the at least one subject-related covariate comprises at least one fetus-related covariate comprising at least one of: gender; fetal weight during pregnancy; fetal biometric parameters such as femur length, abdominal circumference, head circumference, mid-thigh circumference and biparietal diameter; rates of small for gestational age; gestational age; heart rate, heart rate variability; respiration rate; uteroplacental perfusion parameters; and at least one measurement of the at least one biomarker.
M16. The method according to any of the preceding method embodiments and with features of embodiment M8, wherein the at least one subject-related covariate comprises at least one neonate-related covariate, comprising at least one of: gender; birth weight; body weight; body length; gestational age at birth; postnatal age; temperature; heart rate; heart rate variability; respiration rate; breastfeeding; exclusive breastfeeding duration; pH value; breath aid; oxygen demand; blood oxygen saturation (SpO2); blood pressure (systolic/diastolic); Apgar scores; at least additional neonatal biometric parameters; and at least one measurement of the at least one biomarker.
M17. The method according to any of the preceding method embodiments, wherein the at least one subject-related covariate comprises at least one environmental covariate comprising at least one of: country of residence; country of birth; day and time of birth; humidity conditions at birth; and surrounding temperature at birth.
M18. The method according to any of the preceding method embodiments, wherein the method comprises generating at least one threshold, wherein the at least one threshold expresses an indication of at least one potential medical condition.
M19. The method according to any of the preceding method embodiments, wherein the method comprises outputting at least one potential medical condition, wherein the at least one potential medical condition comprises at least one of: seizures; respiratory; cardiovascular; hematological dysfunction; endocrine; renal; hepatic; uteroplacental dysfunction; fetal growth restriction; unplanned preterm birth; placental abruption; hemolysis elevated liver enzymes, low platelets (HELLP) syndrome; and eclampsia.
M20. The method according to any of the preceding method embodiments, wherein the method comprises
   determining a minimum threshold value for the at least one threshold, and
   determining a maximum threshold value for the at least one threshold.
M21. The method according to the preceding embodiment, wherein when the at least one subject-related data is under the minimum threshold value the method comprises outputting a monitoring suggestion.
M22. The method according to any of the preceding 2 embodiments, wherein when the at least one subject-related data is above the maximum threshold value, the method comprises outputting a treatment suggestion.
M23. The method according to any of the 3 preceding embodiments, wherein the method comprises determining a baseline for the at least one subject-related data.
M24. The method according to any of the 4 preceding embodiments, wherein the method comprises at least one intermediate threshold value, wherein the at least one intermediate threshold value comprises at least one value between the minimum threshold value and the maximum threshold value.
M25. The method according to the preceding embodiment, wherein the method comprises
   correlating at least a range of each of the least one intermediate threshold value to the at least one medical condition,
   generating an interpreted dataset based on the correlation step, and
   outputting an automated report indicating the at least one potential medication condition.
M26. The method according to any of the preceding method embodiments, wherein the method comprises
   determining at least one medical condition change indicator,
   monitoring changes of the at least one medical condition change indicator,
   generating at least one medical condition change indicator trend, and
   predicting an evolution of at least one of the at least one medical condition, wherein the predicting is based on the at least one medical condition change indicator trend.
M27. The method according to any of the preceding method embodiments, wherein the method comprises monitoring at least one value change of the at least one subject-related property, the method comprising
   recording an initial value of the at least one subject-related property,
   recording at least one subsequent value of the at least one subject-related property,
   contrasting the initial value with at least one of the at least one subsequent value,
   generating a compared value data, and
   outputting a subject-related property hypothesis based on the compared value data.
M28. The method according to the preceding embodiment, wherein the step of recording at least subsequent value comprises recording one current value of the at least one subject-related property, wherein the current value is different from the initial value.
M29. The method according to any of the preceding method embodiments, wherein the method is a non-diagnostic method.
M30. The method according to any of the preceding method embodiments, wherein the method is a diagnostic method.
M31. The method according to any of the preceding method embodiments, wherein the method comprises carrying out the method steps according to any of the preceding embodiment using data from at least one database.
M32. The method according to the preceding embodiment, wherein the at least one database comprises at least one of: public health database, subject's individual database, medical professional's database, healthcare provider's database, and private databanks.
M33. The method according to any of the preceding method embodiments, wherein the method comprises
   feeding data to the at least one server,
   training the computer-implemented dynamic model based on data fed to the at least one server, and
   generating an adjusting function based on the training data,
   wherein the adjusting function is suitable for adjusting any steps of the method according to any of the preceding method embodiments.
M34. The method according to any of the preceding method embodiments, wherein the method comprises triggering at least one action suggestion based on the at least one health status hypothesis.
M35. The method according to the preceding embodiment, wherein the method comprises displaying the at least one action suggestion to a user.
M36. The method according to any of the 2 preceding embodiments, wherein the method comprises prompting the user to input at least one of acceptation of at least one of the at least one action suggestion, and rejection of at least one of the at least one action suggestion.
M37. The method according to the preceding embodiment, wherein when the user rejects at least one the at least one action suggestion, the method comprises prompting the user to provide at least one annotation.
M38. The method according to any of the preceding method embodiments, wherein the computer-implemented dynamic model is based on a Bayesian statistical approach.
M39. The method according to any of the preceding method embodiments, wherein the computer-implemented dynamic model is based on an ANN, CNN, or RNN approach.
M40. The method according to any of the preceding method embodiments, wherein the computer-implemented dynamic model is based on a pharmacometrics modeling and/or simulation approach.
M41. The method according to any of the preceding method embodiments, wherein the computer-implemented dynamic model is based on a supervised learning approach.
M42. The method according to any of the preceding method embodiments, wherein the computer-implemented dynamic model is based on a deep learning and/or multi-layer neural network approach.
M43. The method according to any of the preceding method embodiments, wherein the computer-implemented dynamic model is based on an explainable AI concept (XAI).
M44. The method according to any of the preceding method embodiments, wherein the at least one medical condition comprises at least one of: fetal growth-related condition; PE-related condition; gestational diabetes-related condition; gestational hypertension-related condition; gestational medical treatment such as cyclooxygenase inhibitors, e.g., Aspirin; at least one relevant medication; and at least one potential medical condition.
M45. The method according to any of the preceding method embodiments and with features of embodiment M13, wherein the method comprises correlating the at least one biomarker to at least one medical condition, wherein the at least one medical condition comprises a potential disease.
M46. The method according to any of the preceding method embodiments, wherein the method comprises predicting occurrence of the at least one hypothesis at a given time period, wherein the method further comprises recognizing a plurality of different time periods comprising at least one of: prenatal period, pregnancy, delivery period, and postnatal period.
M47. The method according to the preceding embodiment, wherein the method comprises outputting a likelihood of occurrence correlated to each of the time periods.
M48. The method according to any of the preceding method embodiment, wherein the method comprises executing at least one machine learning algorithm.
M49. The method according to the preceding embodiment, wherein the at least one machine learning algorithm comprises a supervised algorithm architecture, an unsupervised algorithm architecture, or of any combination thereof.
M50. The method according to any of the preceding method embodiments, wherein the at least one machine learning algorithm comprises at least one artificial deep learning (DL) architecture.
M51. The method according to the preceding embodiment, wherein the at least one artificial deep learning architecture comprises at least one of: ANN, CNN, and RNN.
M52. The method according to any of the preceding method embodiments and with features of embodiment M49, wherein the unsupervised algorithm architecture comprises implementing at least one clustering approach of at least one cluster.
M53. The method according to any of the preceding method embodiments, wherein the method comprises executing at least one analytical approach, wherein the at least one analytical approach comprises at least one of pattern recognition, probabilistic modeling, Bayesian schemes, reinforcement learning, statistical analytics, statistical models, principal component analysis, independent component analysis, dynamic time warping, maximum likelihood estimates, modeling, estimating, neural network, convolutional network, recurrent network, deep convolutional network, deep learning, ultra-deep learning, genetic algorithms, Markov models, and/or hidden Markov models.
M54. The method according to any of the preceding method embodiments, wherein the method comprises implementing at least one pharmacometrics model.
M55. The method according to the preceding embodiment, wherein the at least one pharmacometrics model comprises a computer-implemented pharmacometrics model comprising at least one of: mathematical-statistical PKPD model; physiology-based PK (PBPK) model; physiology-based PKPD (PBPKPD) model; drug exposure-efficacy response model; and drug exposure-safety response model.
M56. The method according to any of the preceding method embodiments, wherein the at least one health status of the subject comprises PE.
M57. The method according to any of the preceding method embodiments, wherein the at least one health status of the subject comprises gestational diabetes.
M58. The method according to any of the preceding method embodiments, wherein the at least one health status of the subject comprises fetal growth-related conditions.
M59. The method according to any of the preceding method embodiments, wherein the at least one health status of the subject comprises gestational hypertension-related conditions.
M60. The method according to any of the preceding method embodiments, wherein step of predicting the at least one health status based on the at least one health status hypothesis, comprises using at least one fetal growth-related data.
M61. The method according to any of the preceding method embodiments, wherein step of predicting the at least one health status comprises using at least one fetal growth-related data, wherein the at least one health status hypothesis is based on the at least one fetal growth-related data.
M62. The method according to the preceding embodiment and with features of embodiment M32, wherein the at least one fetal growth-related data is retrieved from at least one of the at least one database.
M63. The method according to any of the preceding method embodiments, wherein step of predicting the at least one health status comprises using at least one PE-related data, wherein the at least one health status hypothesis is based at least one PE-related data.
M64. The method according to the preceding embodiment and with features of embodiment M32, wherein the at least one PE-related data is retrieved from at least one of the at least one database.
M65. The method according to any of the preceding embodiments, wherein the method comprises determining at least one drug based on the at least one health status, wherein the at least one drug is suitable for preventing occurrence of the at least one medical condition and/or the at least one health status.
M66. The method according to any of the preceding embodiments, wherein the method comprises determining at least one drug based on the at least one health status, wherein the at least one drug is suitable for treating the at least one medical condition and/or the at least one health status.
M67. The system according to any of the 2 preceding embodiments, wherein the at least one drug comprising at least one of: Aspirin; Ibuprofen; at least one corticosteroid drug; at least one anti-hypertensive drug; and at least one cardiovascular-related drug.
M68. The method according to any of the 3 preceding embodiments, wherein the method comprises generating at least one drug administration route, wherein the at least one drug administration route comprises at least one of: intravenous; intramuscular; subcutaneous; inhalation; transdermal; transcutaneous; oral; rectal; and sublingual.
M69. The method according to any of the preceding embodiments and with features of embodiments M45 and M65 to M68, wherein the method comprises generating at least one dosing regimen of the at least one drug.
M70. The method according to the preceding embodiment, wherein the method further comprises optimizing the at least one dosing regimen, wherein the at least one dosing regimen comprises at least one of
   the at least one drug,
   the at least one drug administration route,
   at least one dose scheme,
   at least one drug administration duration, and
   at least one drug administration frequency.
M71. The method according to the preceding embodiment, wherein the step of optimizing the at least one dosing regimen is based on the at least one health status hypothesis.
M72. The method according to any of the 2 preceding embodiments, wherein the method comprises implementing at least optimal control theory, wherein the at least one optimal control theory is computer-implemented.
M73. The method according to any of the preceding method embodiments, wherein the method is a computer-implemented method.
M74. The method according to any of the preceding method embodiments, wherein the method comprises optimizing at least one ongoing treatment of the at least one medical condition.
M75. The method according to any of the preceding method embodiments, wherein the method comprises optimizing at least one ongoing treatment of the at least one potential medical condition.
M76. The method according to any of the preceding 2 embodiments, wherein the step of optimizing is based the at least one health status hypothesis and/or the at least one health status.
M77. The method according to any of the preceding method embodiments, wherein the method comprises generating at least one treatment suggestions, wherein the at least one treatment suggestion is based on the at least one health status hypothesis and/or the at least one health status.
M78. The method according to the preceding embodiment, wherein the method comprises optimizing the at least treatment suggestion, wherein the method comprises carrying out the optimizing step after executing the at least one treatment suggestion.
M79. The method according to any of the preceding method embodiments, wherein the method comprises
   adapting any of the preceding method embodiments to the subject, and
   generating at least one individualized treatment protocol, wherein the at least one individualized treatment protocol is based on the at least one health status of the subject.
M80. The method according to the preceding embodiment, wherein the method comprises optimizing the at least individualized treatment protocol, wherein comprises carrying out the optimizing step after executing the at least one individualized treatment protocol.
M81. The method according to any of the preceding embodiments, wherein the method comprises adapting any of the preceding method embodiments to the subject via online learning.
M82. The method according to any of the preceding method embodiments, wherein the method comprises implanting any of the preceding optimizing steps assisted by a computer-implemented pharmacometrics approach.
M83. The method according to any of the preceding embodiments, wherein the method comprises carrying out any of the preceding method embodiments in absence of the subject.
M84. The method according to any of the preceding embodiments, wherein the method comprises carrying out any of the preceding method embodiments using at least one historical data.
M85. The method according to the preceding embodiment, wherein the at least one historical data is a historical data of the subject.
M86. The method according to any of the 2 preceding embodiments, wherein the at least one historical data comprises data from at least one of
   public health database
   subject's individual database,
   medical professional's database,
   healthcare provider's database, and
   private databanks.
M87. The method according to any of the preceding method embodiments, wherein the method comprises at least one of
   capturing at least one image data of the subject; and
   receiving at least one image data of the subject,
wherein the at least one image data comprises data relevant to at least medical condition and/or at least one potential medical condition of the subject.
M88. The method according to any of the preceding embodiments, wherein the method is suitable for implementation in at least one medical device such as an ultrasound device.
M89. The method according to any of the preceding method embodiments, wherein the method comprises using the system according to any of the system embodiments to perform any of the steps according to any of the method embodiments.
M90. The method according to any of the preceding method embodiments, wherein the method is a method for continuously predicting the health status of a subject and/or an ambulatory subject.
M91. The method according to any of the preceding embodiments, wherein the method comprises individualizing the predicting according to at least one data related to the subject.
M92. The method according to any of the preceding embodiments with the features of embodiment M2, wherein the predicting of the at least one health status based on a computer-implemented dynamic model, is based on the generation of the at least one health status of the subject.

M93. The method according to any of the preceding embodiments, wherein the method comprises synchronizing the at least one subject-related covariate and/or the at least one subject-related property data with respect to time.
M94. The method according to any of the preceding embodiments, wherein the method comprises synchronizing the at least one subject-related covariate and/or the at least one subject-related property data according to at least one timestamp comprised in the at least one subject-related covariate and/or at least one subject-related property data.
M95. The method according to any of the preceding embodiments with the features of embodiment M94, wherein the at least one timestamp represents the date and/or time the at least one subject-related covariate and/or the at least one subject-related property data was recorded.
M96. The method according to any of the preceding embodiments with the features of embodiment M94, the at least one timestamp is recorded with a resolution in the range of seconds, minutes, hours, days, weeks and/or months.
M97. The method according to any of the preceding embodiments with the features of embodiment M94, wherein the method comprises modifying the resolution of the timestamp of the at least one subject-related covariate and/or the at least one subject-related property data.
M98. The method according to any of the preceding embodiments with the features of embodiment M94, wherein the method comprises modifying the resolution of the timestamp of the at least one subject-related covariate and/or the at least one subject-related property data, into a standardized timestamp resolution.
M99. The method according to any of the preceding embodiments, wherein the method comprises modifying the impact of the at least one subject-related covariate and/or the at least one subject-related property data, on at least one output of the method, according to the timestamp and/or according to the at least one subject-related covariate and/or the at least one subject-related property data.
M100. The method according to any of the preceding embodiments, wherein the method comprises synchronizing the at least one subject-related covariate and/or the at least one subject-related property data with respect to each other.
M101. The method according to any of the preceding embodiments, wherein the method comprises synchronizing the at least one subject-related covariate and/or the at least one subject-related property data relatively to at least one data, wherein the at least one data may comprise at least one subject-related covariate and/or at least one subject-related property data.
M102. The method according to any of the preceding embodiments, wherein the method comprises converting the data type of the at least one subject-related covariate and/or the at least one subject-related property data, into a standardized data type.
M103. The method according to any of the preceding embodiments, wherein the method comprises removing at least one outlier comprised in the at least one subject-related covariate and/or the at least one subject-related property data.
M104. The method according to any of the preceding embodiments with the features of embodiment M36, wherein the method comprises utilizing at least one acceptation and/or the at least one rejection for optimizing at least one algorithm implemented by the method.
M105. The method according to any of the preceding embodiments with the features of embodiment M36, wherein the method comprises utilizing at least one acceptation and/or the at least one rejection for reinforcement learning.
M106. The method according to any of the preceding embodiments with the features of embodiment M36, wherein the method comprises utilizing at least one acceptation and/or the at least one rejection for online learning.
M107. The method according to any of the preceding method embodiments, wherein the computer-implemented dynamic model is based on a reinforcement learning approach.
M108. The method according to any of the preceding method embodiments, wherein the computer-implemented dynamic model is based on a hybrid approach.
M109. The method according to any of the preceding method embodiments, wherein the
   computer-implemented dynamic model is based at least one on decision tree approach and/or at least one random forests approach.

Below, system embodiments will be discussed. These embodiments are abbreviated by the letter “S” followed by a number. When reference is herein made to a system embodiment, those embodiments are meant.
S1. A system for predicting health status of a subject, the system comprising
   at least one processing component configured to
      receive at least one subject-related dynamic property data,
      receive at least one subject-related covariate, and
      process the at least one subject-related dynamic property data and the at least one subject-related covariate data to generate a subject-related processed dataset,
   at least one analyzing component configured to
      analyze the subject-related processed dataset, and
      generate at least one health status hypothesis based on the subject-related processed dataset,
wherein the system is configured to predict at least one health status based on the at least one health status hypothesis.
S2. The system according to the preceding embodiment, wherein the system comprises at least one at least one storing component configured to store data relevant to the at least one health status of the subject.
S3. The system according to the preceding embodiment, wherein the system comprises at least one computing component configured to implement dynamic model for predicting the at least one health status.
S3. The system according to any of the preceding system embodiments, wherein the at least one health status hypothesis comprises a correlation to at least one medical condition of the subject.
S4. The system according to any of the preceding system embodiments, wherein the subject is a female subject.
S5. The system according to the preceding embodiment, wherein the female subject is a pregnant woman.
S6. The system according to any of the 2 preceding embodiments, wherein the female subject comprises a non-pregnant woman.
S7. The system according to any of the preceding system embodiments, wherein the subject is a fetus.
S8. The method according to any of the preceding method embodiments, wherein the subject is a neonate.
S9. The system according to any of the preceding system embodiments, wherein the system is configured to predict a dynamic behavior of the at least one health status of the subject.
S10. The system according to any of the preceding system embodiments, wherein the system is configured to implement at least one machine learning technique, wherein the system is configured to perform any of the steps according to any of the preceding method embodiments by means of the at least one machine learning technique.
S11. The system according to any of the preceding system embodiments, wherein the at least one subject-related covariate comprises at least one biomarker.
S12. The system according to the preceding embodiment, wherein the at least one biomarker is related to at least one medical condition.
S13. The system according to any of the 3 preceding embodiments, wherein the at least one biomarker comprises at least one of: soluble Fms-like tyrosine kinase-1 (sFlt-1); placental growth factor (PIGF); neurofilament light chain (NfL); Copeptin; Placental biomarkers such as placental RNAs, placental proteins; Endothelial/cardiovascular biomarkers such as endothelial RNAs, endothelial proteins; or any combination thereof.
S14. The system according to any of the preceding system embodiments and with features of embodiment S4, wherein the at least one subject-related covariate comprises at least one neonate-related covariate comprising at least one of: gender, race, birth weight, gestational age, birth mode such as vaginal, vacuum extraction, cesarean, temperature, heart rate, respiration rate, pH value, umbilical cord pH value, breath aid, oxygen demand, blood oxygen saturation (SpO2), blood pressure (systolic/diastolic), Apgar scores, and at least one measurement of the at least one biomarker.
S15. The system according to any of the preceding system embodiments and with features of embodiment S5, wherein the at least one subject-related covariate comprises at least one mother-related covariate comprising at least one of: age, race, early membrane rupture, temperature, risk factors such as diabetes, adiposity, gravidity, parity, leukocytes, and at least one measurement of the at least one biomarker.
S16. The system according to any of the preceding system embodiments and with features of embodiment S6, wherein the at least one subject-related covariate comprises at least one fetus-related covariate comprising at least one of: gender; fetal weight during pregnancy; fetal biometric parameters such as femur length, abdominal circumference, head circumference, mid-thigh circumference and biparietal diameter; rates of small for gestational age; gestational age; heart rate, heart rate variability; respiration rate; utero-placental perfusion parameters; and at least one measurement of the at least one biomarker.
S17. The system according to any of the preceding system embodiments, wherein the at least one subject-related covariate comprises at least one environmental covariate comprising at least one of: country of residence, country of birth, day and time of birth, humidity conditions at birth, and surrounding temperature at birth.
S18. The system according to any of the preceding system embodiments, wherein the system is configured to generate at least one threshold, wherein the at least one threshold expresses an indication of at least one potential a medical condition.
S19. The system according to any of the preceding system embodiments, wherein the system is configured to output at least one potential medical condition, wherein the at least one potential medical condition comprises at least one of: seizures; respiratory; cardiovascular; hematological dysfunction; endocrine; renal; hepatic; uteroplacental dysfunction; fetal growth restriction; unplanned preterm birth; placental abruption; hemolysis elevated liver enzymes, low platelets (HELLP) syndrome; and eclampsia.
S20. The system according to any of the preceding system embodiments, wherein the at least one analyzing component is configured to
   determine a minimum threshold value for the at least one threshold, and
   determine a maximum threshold value for the at least one threshold.
S21. The system according to the preceding embodiment, wherein when the at least one subject-related data is under the minimum threshold value the at least one analyzing component outputs a monitoring suggestion.
S22. The system according to any of the preceding 2 embodiments, wherein when the at least one subject-related data is above the maximum threshold value, the at least one analyzing component outputs a treatment suggestion.
S23. The system according to any of the 3 preceding embodiments, wherein the at least one analyzing component is configured to determining a baseline for the at least one subject-related data.
S24. The system according to any of the 4 preceding embodiments, wherein the at least one analyzing component is configured to determine at least one intermediate threshold value, wherein the at least one intermediate threshold comprises at one value between the minimum threshold value and the maximum threshold value.
S25. The system according to the preceding embodiment, wherein the at least one analyzing component is configured to
   correlate at least a range of each of the least one intermediate is correlated to at least one medical condition,
   generate an interpreted dataset based on the correlation step, and
   output an automated report indicating at least one potential medication condition.
S26. The system according to any of the preceding system embodiments, wherein the at least one analyzing component is configured to
   determine at least one medical condition change indicator,
   monitor changes of the at least one medical condition change indicator,
   generate at least one medical condition change indicator trend, and
   predict an evolution of at least one of the at least one medical condition based on the at least one medical condition change indicator trend.
S27. The system according to any of the preceding system embodiments, wherein the system comprises at least one monitoring component configured to monitor at least one value change of the at least one subject-related property, wherein the at least one monitoring component is further configured to
   record an initial value of the at least one subject-related property,
   record at least one subsequent value of the at least one subject-related property,
   contrast the initial value with at least one of the at least one subsequent value,
   generate a compared value data, and
   output a subject-related property hypothesis based on the compared value data.
S28. The system according to the preceding embodiment, wherein the at least one monitoring component is configured to record one current value of the at least one subject-related property, wherein the current value is different from the initial value.
S29. The system according to any of the preceding system embodiments, wherein the system is a non-diagnostic system.
S30. The system according to any of the preceding system embodiments, wherein the system is a diagnostic system.
S31. The system according to any of the preceding system embodiments, wherein the system is configured to carry out the method steps according to any of the preceding method embodiments using data from at least one database.
S32. The system according to the preceding embodiment, wherein the at least one database comprises at least one of: public health database, subject's individual database, medical professional's database, healthcare provider's database, and private databanks.
S33. The system according to any of the preceding system embodiments, wherein the system is configured to
   feed data to the at least one server, and
   train the computer-implemented dynamic model based on data fed to the at least one server, and
   generate an adjusting function based on the training data,
   wherein the adjusting function is suitable for adjusting any configuration of the system according to any of the preceding system embodiments.
S34. The system according to any of the preceding system embodiments, wherein the system is configured to trigger at least one action suggestion based on the at least one health status hypothesis.
S35. The system according to the preceding embodiment, wherein the system is configured to display the at least one action suggestion to a user.
S36. The system according to any of the 2 preceding embodiments, wherein the system is configured to prompt the user to input at least one of
   acceptation of at least one of the at least one action suggestion, and
   rejection of at least one of the at least one action suggestion.
S37. The system according to the preceding embodiment, wherein when the user rejects at least one the at least one action suggestion, the system is configured to prompt the user to provide at least one annotation.
S38. The system according to any of the preceding system embodiments, wherein the computer-implemented dynamic model is based on a Bayesian statistical approach.
S39. The system according to any of the preceding system embodiments, wherein the computer-implemented dynamic model is based on an ANN, CNN, or RNN approach.
S40. The system according to any of the preceding system embodiments, wherein the computer-implemented dynamic model is based on a pharmacometrics approach.
S41. The system according to any of the preceding system embodiments, wherein the computer-implemented dynamic model is based on a supervised learning approach.
S42. The system according to any of the preceding system embodiments, wherein the computer-implemented dynamic model is based on a deep learning and/or multi-layer neural network approach.
S43. The system according to any of the preceding system embodiments, wherein the computer-implemented dynamic model is based on an explainable Al concept (XAI).
S44. The system according to any of the preceding system embodiments, wherein the at least one medical condition comprises at least one of: fetal growth-related condition; neonatal thyroid dysfunction; PE-related condition; gestational diabetes related condition; gestational hypertension-related condition; and gestational thyroid dysfunction.
S45. The system according to any of the preceding system embodiments and with features of embodiment S13, wherein the system is configured to correlate the at least one biomarker to at least one medical condition, wherein the at least one medical condition comprises a potential disease.
S46. The system according to any of the preceding system embodiments, wherein the system is configured to predict occurrence of the at least one hypothesis at a given time period, wherein the system is configured to recognize a plurality of different time periods comprising at least one of: prenatal period, pregnancy, delivery period, and postnatal period.
S47. The system according to the preceding embodiment, wherein the system is configured to output a likelihood of occurrence correlated to each of the time periods.
S48. The system according to any of the preceding system embodiment, wherein the system is configured to execute at least one machine learning algorithm.
S49. The system according to the preceding embodiment, wherein the at least one machine learning algorithm comprises a supervised algorithm architecture, an unsupervised algorithm architecture, or of any combination thereof.
S50. The system according to any of the preceding system embodiments, wherein the at least one machine learning algorithm comprises at least one artificial deep learning (DL) architecture.
S51. The system according to the preceding embodiment, wherein the at least one artificial deep learning architecture comprises at least one of: ANN, CNN, and RNN.
S52. The system according to any of the preceding system embodiments and with features of embodiment S47, wherein the unsupervised algorithm architecture is configured to implement at least one clustering approach of at least one cluster.
S53. The system according to any of the preceding system embodiments, wherein the system is configured to execute at least one analytical approach, wherein the at least one analytical approach comprises at least one of pattern recognition, probabilistic modeling, Bayesian schemes, reinforcement learning, statistical analytics, statistical models, principal component analysis, independent component analysis, dynamic time warping, maximum likelihood estimates, modeling, estimating, neural network, convolutional network, recurrent network, deep convolutional network, deep learning, ultra-deep learning, genetic algorithms, Markov models, and/or hidden Markov models.
S54. The system according to any of the preceding system embodiments, wherein the system is configured to implement at least one pharmacometrics model.
S55. The system according to the preceding embodiment, wherein the at least one pharmacometrics model comprises a PKPD model.
S56. The system according to any of the preceding system embodiments, wherein the at least one health status of the subject comprises PE.
S57. The system according to any of the preceding system embodiments, wherein the at least one health status of the subject comprises gestational diabetes.
S58. The system according to any of the preceding system embodiments, wherein the at least one health status of the subject comprises fetal growth-related issues.
S59. The system according to any of the preceding system embodiments, wherein the at least one health status of the subject comprises gestational hypertension-related conditions.
S60. The system according to any of the preceding system embodiments, wherein the system is configured to predict the at least one health status based on the at least one health status hypothesis, and use at least one fetal growth-related data.
S61. The system according to any of the preceding system embodiments, wherein the system is configured to predict the at least one health status using at least one fetal growth-related data, wherein the at least one health status hypothesis is based on the at least one fetal growth-related data.
S62. The system according to the preceding embodiment and with features of embodiment S30, wherein the at least one fetal growth-related data is retrieved from at least one of the at least one database.
S63. The system according to any of the preceding system embodiments, wherein the system is configured to predict the at least one health status comprises using at least one PE-related data, wherein the at least one health status hypothesis is based at least one PE-related data.
S64. The system according to the preceding embodiment and with features of embodiment S30, wherein the at least one PE-related data is retrieved from at least one of the at least one database.
S65. The system according to any of the preceding system embodiments, wherein the system comprises at least one imaging component configure to at least one of
   capture at least one image data of the subject; and
   receive at least one image data of the subject,
wherein the at least one image data comprises data relevant to at least medical condition and/or at least one potential medical condition of the subject.
S66. The system according to any of the preceding system embodiments, wherein the system is configured to perform any of the steps according to any of the method embodiments.
S67. The system according to any of the preceding system embodiments, wherein the system comprises at least one implementation component configured to connect the system to at least one medical device such as an ultrasound device, wherein the system once connected to the at least one medical device is configured to perform any of the steps according to any of the preceding method embodiments.
S68. The system according to any of the preceding system embodiments, wherein the system is configured to operate in absence of the subject.
S69. The system according to any of the preceding system embodiments, wherein the system comprises at least one implementation component configured to connect the system to at least one medical device such as a sensor, wherein the system once connected to the at least one medical device is configured to perform any of the steps according to any of the preceding method embodiments.
S69. The system according to any of the preceding system embodiments, wherein the system comprises at least one implementation component configured to connect the system to at least one external medical device, wherein the system once connected to the at least one external medical device is configured to perform any of the steps according to any of the preceding method embodiments.
S70. The system according to any of the preceding system embodiments, wherein the components of the system are configured to transmit and/or receive data to and/or from each other regardless of the distance between each component.
S71. The system according to any of the preceding system embodiments, wherein the system is a system for continuously predicting the health status of a subject and/or an ambulatory subject.
S72. The system according to any of the preceding system embodiments, wherein the system is configured to individualize the predicting according to at least one data related to the subject.
S73. The system according to any of the preceding system embodiments, wherein the system is configured to synchronize the at least one subject-related covariate and/or the at least one subject-related property data with respect to time.
S74. The system according to any of the preceding system embodiments, wherein the system is configured to synchronize the at least one subject-related covariate and/or the at least one subject-related property data according to at least one timestamp comprised in the at least one subject-related covariate and/or at least one subject-related property data.
S75. The system according to any of the preceding system embodiments with the features of embodiment S74, wherein the at least one timestamp represents the date and/or time the at least one subject-related covariate and/or the at least one subject-related property data was recorded.
S76. The system according to any of the preceding system embodiments with the features of embodiment S74, wherein the at least one timestamp is recorded with a resolution in the range of seconds, minutes, hours, days, weeks and/or months.
S77. The system according to any of the preceding system embodiments with the features of embodiment S74, wherein the system is configured to modify the resolution of the timestamp of the at least one subject-related covariate and/or the at least one subject-related property data.
S78. The system according to any of the preceding system embodiments with the features of embodiment S74, wherein the system is configured to modify the resolution of the timestamp of the at least one subject-related covariate and/or the at least one subject-related property data, into a standardized timestamp resolution.
S79. The system according to any of the preceding system embodiments, wherein the system is configured to modify the impact of the at least one subject-related covariate and/or the at least one subject-related property data, on at least one output of the system, according to the timestamp and/or according to the at least one subject-related covariate and/or the at least one subject-related property data.
S80. The system according to any of the preceding system embodiments, wherein the system is configured to synchronize the at least one subject-related covariate and/or the at least one subject-related property data with respect to each other.
S81. The system according to any of the preceding system embodiments, wherein the system is configured to synchronize the at least one subject-related covariate and/or the at least one subject-related property data relatively to at least one data, wherein the at least one data may be configured to at least one subject-related covariate and/or at least one subject-related property data.
S82. The system according to any of the preceding system embodiments, wherein the system is configured to convert the data type of the at least one subject-related covariate and/or the at least one subject-related property data, into a standardized data type.
S83. The system according to any of the preceding system embodiments, wherein the system is configured to remove at least one outlier comprised in the at least one subject-related covariate and/or the at least one subject-related property data.
S84. The system according to any of the preceding system embodiments with the features of embodiment S36, wherein the system is configured to utilize at least one acceptation and/or the at least one rejection for optimizing at least one algorithm implemented by the system.
S85. The system according to any of the preceding system embodiments with the features of embodiment S36, wherein the system is configured to utilize at least one acceptation and/or the at least one rejection for reinforcement learning.
S86. The system according to any of the preceding system embodiments with the features of embodiment S36, wherein the system is configured to utilize at least one acceptation and/or the at least one rejection for online learning.
S97. The system according to any of the preceding system embodiments, wherein the
   computer-implemented dynamic model is based on a reinforcement learning approach.
S98. The system according to any of the preceding system embodiments, wherein the
   computer-implemented dynamic model is based on a hybrid approach.
S99. The system according to any of the preceding system embodiments, wherein the
   computer-implemented dynamic model is based on at least one decision tree approach and/or random forests approach.

Below, method of treatment embodiments will be discussed. These embodiments are abbreviated by the letter “T” followed by a number. When reference is herein made to a method of treatment embodiment, those embodiments are meant.
T1. A method of treatment for treating a medical condition of a subject, wherein the treatment comprises generating a treatment protocol comprising at least one treatment drug and a treatment regimen, wherein the treatment regimen is based on at least one health status hypothesis.
T2. The treatment according to the preceding embodiment, wherein the at least one health status hypothesis is provided by the method according to any of the preceding method embodiments.
T3. The treatment according to any of the 2 preceding embodiments, wherein the at least one drug is provided by the method according to any of the preceding method embodiments.
T4. The treatment according to any of the preceding treatment embodiments, wherein the at least one health status of the subject comprises PE.
T5. The treatment according to any of the preceding treatment embodiments, wherein the at least one health status of the subject comprises gestational diabetes and/or gestational hypertension.
T6. The treatment according to any of the preceding treatment embodiments, wherein the at least one health status of the subject comprises fetal growth issues.
T7. The treatment according to any of the preceding treatment embodiments, wherein the treatment further comprises treating the subject for at least one potential medical condition previous to onset of the at least one medical condition.
T8. The treatment according to any of the preceding treatment embodiments, wherein the subject is at least one of: a pregnant woman, and a non-pregnant woman.
T9. The treatment according to any of the preceding treatment embodiments, wherein the subject is a fetus.
T10. The treatment according to any of the preceding treatment embodiments, wherein the subject is a neonate.

Below, diagnostic method embodiments will be discussed. These embodiments are abbreviated by the letter “D” followed by a number. When reference is herein made to a diagnostic embodiment, those embodiments are meant.
D1. A diagnostic method for diagnosing a medical condition of a subject, wherein the diagnosis comprises generating at least one diagnostic finding comprising at least one medical condition of the subject, wherein the at least one diagnostic finding is based on at least one health status hypothesis.
D2. The diagnostic according to the preceding embodiment, wherein the diagnostic comprises generating at least one method of treatment, wherein the at least one method of treating is for treating the at least one medical condition of the subject.
D3. The method according to any of the 2 preceding embodiments, wherein the diagnostic comprises generating the at least one diagnostic finding, wherein the at least at least diagnostic finding comprises the at least one medical condition of the subject previous to onset of the at least one medical condition.
D4. The diagnostic according to the preceding embodiment, wherein the diagnostic comprising at least one preventive method of treatment, wherein the at least one preventive method of treatment is for treating the at least one medical condition of the subject previous to onset of the at least one medical condition.
D5. The diagnostic according to any of the preceding diagnostic embodiments, wherein the at least one health status hypothesis is provided by the method according to any of the preceding method embodiments.
D6. The diagnostic according to any of the preceding diagnostic embodiments, wherein the diagnostic comprises providing at least one drug, wherein the at least one drug is provided by the method according to any of the preceding method embodiments.
D7. The diagnostic according to any of the preceding diagnostic embodiments, wherein the at least one health status of the subject comprises PE.
D8. The diagnostic according to any of the preceding diagnostic embodiments, wherein the at least one health status of the subject comprises gestational diabetes and/or gestational hypertension.
D9. The diagnostic according to any of the preceding diagnostic embodiments, wherein the at least one health status of the subject comprises fetal growth issues.
D10. The diagnostic according to any of the preceding diagnostic embodiments, wherein the subject is at least one of: a pregnant woman, and non-pregnant woman.
D11. The diagnostic according to any of the preceding diagnostic embodiments, wherein the subject is a fetus.
D12. The diagnostic according to any of the preceding diagnostic embodiments, wherein the subject is a neonate.
D13. The diagnostic according to any of the preceding diagnostic embodiments, wherein the diagnostic comprises suggesting a method of treating according to any of the preceding method of treatment embodiments.

Below, use embodiments will be discussed. These embodiments are abbreviated by the letter “U” followed by a number. When reference is herein made to a system embodiment, those embodiments are meant.
U1. Use of the system according to any of the preceding system embodiments for carrying out the method according to any of the preceding method embodiments.
U2. Use of the method according to any of the preceding method embodiments, wherein the method comprises prompting the system according to any of the preceding embodiments to perform the steps of the method according to any of the preceding method embodiments.
U3. Use of the method according to any of the preceding method embodiments for implementing the method of treatment according to any of the preceding method of treatment embodiments.
U4. Use of the method according to any of the preceding method embodiments for implementing the diagnostic method according to any of the preceding diagnostic method embodiments.
U5. Use of the method according to any of the preceding method embodiments for implementing the diagnostic method according to any of the preceding diagnostic method embodiments and the method of treatment according to any of the preceding method of treatment embodiments, wherein implementing the diagnostic method precedes implementing the method of treatment.

The present invention will now be described with reference to the accompanying drawings which illustrate embodiments of the invention. These embodiments should only exemplify, but not limit, the present invention.

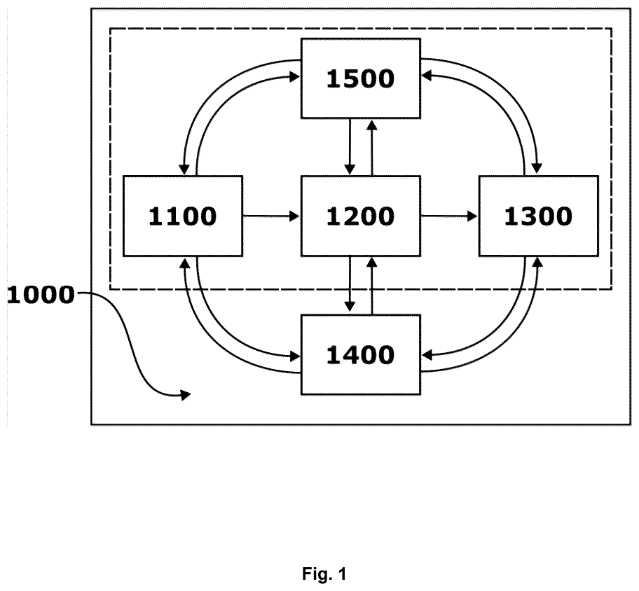

FIG. 1 schematically depicts a system according to embodiments of the present invention for predicting health status of a subject;

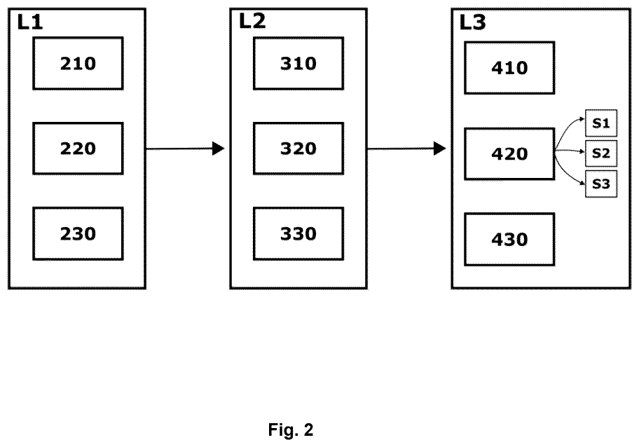

FIG. 2 schematically depicts a layer-like representation of an implementation of invention according to embodiments of the present invention;

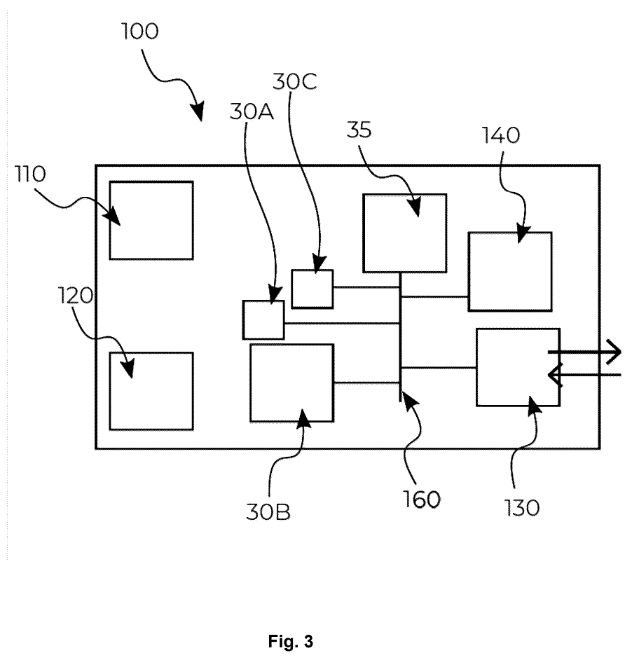

FIG. 3 schematically exemplifies a flowchart in accordance with an embodiment according to the invention,

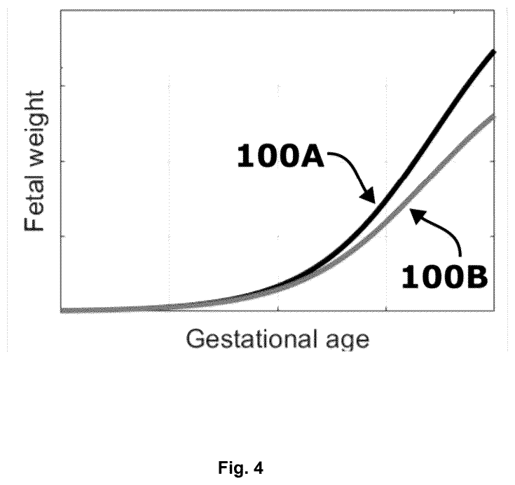

FIG. 4 depicts pregnancy evolution comparison between two types of subjects,

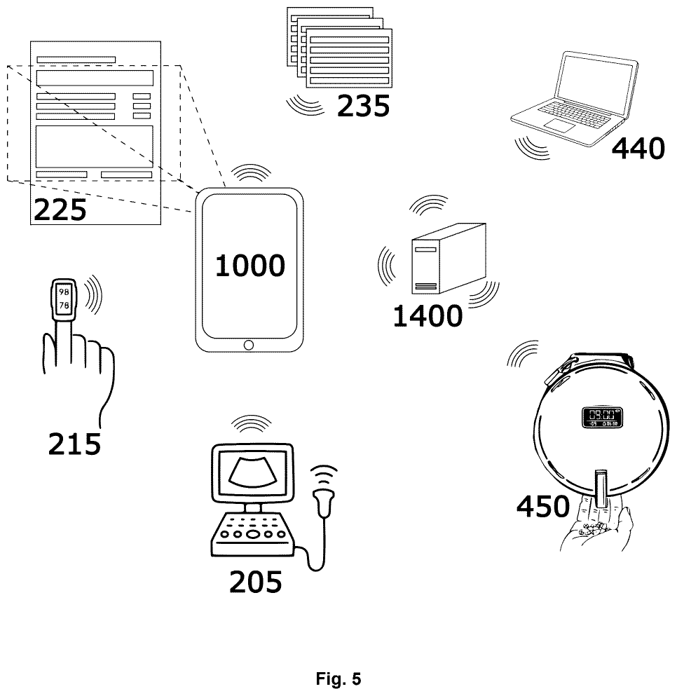

FIG. 5 depicts a schematized example of the system according to embodiments of the present invention.

It is noted that not all the drawings carry all the reference signs. Instead, in some of the drawings, some of the reference signs have been omitted for sake of brevity and simplicity of illustration. Embodiments of the present invention will now be described with reference to the accompanying drawings.

FIG. 1 schematically depicts a system 1000 for predicting health status of a subject. In simple terms the system 1000 comprises a processing component 1100, an analyzing component 1200, a computing component 1300, a storing component 1400 and a monitoring component 1500. It should be understood that in some embodiments, the system 1000 may comprise one or more of these components.

In an embodiment, the storing component 1400 may be an external component, such as a remote component. In FIG. 1 this is denoted by the dashed lines. However, it should be understood that any other component of the system 1000 may also be external, for instance, the monitoring component 1500 may be a remote component. When a component of the system 1000 is an external component, it should be understood that this may also be allocated on a server (remote or local) or even in a cloud.

The processing component 1100 may be configured to receive at least one subject-related dynamic property data, receive at least one subject-related covariate, and process the at least one subject-related dynamic property data and the at least one subject-related covariate data to generate a subject-related processed dataset. That is, the processing component 1100 is charged to receive data, such as, raw data or unprocessed data from a different system such a database, a manual input by a user, an automatic input performed by another device or system. Once the processing component 1100 has received the data, it autonomously or at least partially autonomously can process the data in order to generate subject-related processed datasets.

The analyzing component 1200 may be configured to analyze the subject-related processed dataset and generate at least one health status hypothesis based on the subject-related processed dataset.

In one embodiment of the system 1000, the processing component 1100 and the analyzing component 1200 may represent an integrated component.

The system 1000 is configured to utilize a plurality of different data as input. Inter alia but not limited to, the system 1000 may receive, process and/or analyze a plurality of biomarker such as parental, fetal and/or neonatal biomarkers; a plurality of clinical parameters such as parental, fetal and/or neonatal clinical parameters; demographics, lifestyles and psychometric scores related to the subject and/or a group of subjects; a plurality of environmental parameters; drug treatments such as current drug treatments on the subject and/or recommended drug treatments in guidelines in effect or in force; dosing regimens, drug history related to the subject or a group of subjects; cardiography data (CTG); electroencephalogram data (EEG); electrocardiogram data (ECG), pulse and/or oxygen measurements; data provided, for instance, by sono and variants such as doppler, duplex; magnetic resonance imaging (MRI); X-ray.

In an embodiment, the system 1000 may also comprise one or more imaging component (not depicted) configured to capture images of the subject that may be relevant to the at least one health status and/or a medical condition.

The monitoring component 1500 is configured to monitor the system 1000, i.e., components of the system 1000. Moreover, the monitoring component 1500 may be configured to: monitor at least one value change of the at least one subject-related property; record an initial value of the at least one subject-related property; record at least one subsequent value of the at least one subject-related property; contrast the initial value with at least one of the at least one subsequent value; generate a compared value data, and output a subject-related property hypothesis based on the compared value data.

Additionally or alternatively, the monitoring component 1500 may also be configured to record one current value of the at least one subject-related property, wherein the current value is different from the initial value. That is, the monitoring component 1500 is configured to monitor changes of the value over time of the at least one subject-related property. It should be understood that for this purpose the monitoring component 1500 or the system 1000 or a component of the system 1000 may record and/or determine an initial value. However, it should also be understood that this initial value may already be contained in the received data. In some embodiments, the initial value may also be referred to as baseline.

Moreover, the system 1000 is configured to predict at least one health status of the subject based on the at least one status hypothesis.

The computing component 1300 is configured to implement a dynamic model for predicting the at least one health status. In one embodiment, the computing component 1300 is also configured to implement a plurality of models to predict the at least one health status, to improve a finding, to suggest, generate and/or improve a drug for treatment of the at least one health status.

The storing component 1400 is also configured to store data relevant to the at least one health status of the subject. In one embodiment, the storing component 1400 may also comprise a server comprising a plurality of computer-implemented modules. In a further embodiment, the storing component 1400 may also comprise at least partially the processing component 1100, the analyzing component 1200, the computing component 1400 and/or the monitoring component 1500.

In one embodiment, the computing component 1300 may also comprise a computing device as described further in FIG. 3.

The system is further configured to output a plurality of data comprising information related to the subject such as PE. This information, inter alia, may comprise onset data, severity data scoring and prediction, onset dynamic analysis and interpretation, risk assessment of the subject such as a mother, a fetus, or a neonate. The risk assessment may further comprise prediction and/or estimation of maternal, fetal and/or neonatal complications, type of complications and/or level of complications. Moreover, the system 1000 is configured to output at least one therapeutic suggestion and/or a therapeutic protocol and/or the optimization of an ongoing therapeutic protocol.

In one embodiment, the system 1000 may also comprise a signal processing component (not depicted) configured to process a plurality of signals supplied one or more devices external and/or independent from the system 1000. The signal processing component may also be comprised by the processing component 1100 and configured to process data received as signal data.

For example, within 12-16 weeks of pregnancy, during a routine check-up, the medical professional may record key subject data such as family and pregnancy history, vital signs (e.g., weight, blood pressure), and routine lab values (e.g., creatinine, sodium). These inputs may partially be entered into the system, which then may generate a personalized risk score for the likelihood of the patient developing the targeted condition. Based on the results, the medical professional may decide on the appropriate course of action. This may or may not comprise turning the subject into an ambulatory subject.

While the ambulatory subject is monitored outside of a medical care facility, the system continuously may analyze incoming data and may trigger automated alerts to the medical professional if the ambulatory subject's risk level changes due to evolving individual circumstances.

Another example would be, after the 20^{th} week of pregnancy, during another routine check-up, the medical professional inputs advanced biomarkers along with other disease-specific symptoms into the system. The system provides a detailed risk score indicating the likelihood of adverse outcomes during the remainder of the pregnancy. Additionally, it calculates the probability of the patient delivering within a specified time frame, helping the doctor tailor further care.

From the subject's perspective, the system guides the subject through every step of the process. Data collected at home, such as vitals measured by integrated devices, is automatically transmitted to the medical professional. This provides the subject with a heightened sense of safety and reassurance. The onboarding process is automated, and continuous monitoring is straightforward, ensuring the system is easy to use while offering valuable insights.

Said insights may comprise, for example, a risk profile prediction of pre-eclampsia for the patient. The system may thus provide the subject with a treatment in the form of, for example, a certain combination and quantity of various medications from a pill dispenser, comprising multitple types of pills, according to the diagnosis which may depend on the multiple factors inputted such as but not limited to the subject's weight, blood pressure, and/or medical history.

Subject B, for example, at 14 weeks of pregnancy, attends a routine check-up. During the visit, the following data is recorded and entered into the system: The subject's age (32 years old), the subject's BMI (28), blood pressure (135/85 mmHg). The subject's family history of pre-eclampsia, disclosing that the subject's mother experienced pre-eclampsia during two pregnancies, is also recorded into the system, as well as the patient's own pregnancy history, which in this example, includes a prior pregnancy with gestational hypertension. Additional lab results were also be inputted into the system comprising serum creatinine of 0.9 mg/dL, sodium levels of 138 mEg/L and Negative Proteinuria. The system processes these inputs using a dynamic model and generates a personalized risk score. Patient B is identified as having an elevated risk for developing preeclampsia. Based on this score, the system generates a treatment as preventative measure including low-dose aspirin (150 mg) to be taken daily, recommendations for regular blood pressure monitoring at home using a blood pressure cuff for example, and outputs a warning message to the corresponding medical professional and the subject regarding follow-up visits recommended every four weeks for closer observation.

Subject B is thus monitored at home using the system acquiring data from wearble devices such as the blood pressure cuff. Data from the wearable device is transmitted automatically to the system in real-time: blood pressure readings, which in this case comprise average weekly values ranging between 140/90 mmHg and 145/90 mmHg, weight gain which is found to be consistent with pregnancy norms and a normal fetal heart rate. At 20 weeks, Patient B experiences mild headaches and reports these symptoms through the system's patient interface. The system's algorithm flags this as a potential indicator of worsening preeclampsia risk and generates an alert for the medical professional. An interim visit is scheduled.

During the follow-up visit, at 28 weeks of pregnancy, the following evaluations are ordered by the medical professional and the data acquired from these evaluations is inputted into the system: symptoms of the patient comprising persistent headaches and visual disturbances and swelling in hands and feet, blood pressure of 155/100 mmHg, Proteinuria detected (protein: creatinine ratio of 350 mg/mmol), Elevated sFlt-1/PIGF ratio of 125, Platelet count of 110,000/μL, Ultrasound revealing reduced amniotic fluid (AFI: 6 cm), and abnormal Doppler findings comprising elevated umbilical artery pulsatility index. The system consolidates these data points and confirms a diagnosis of preeclampsia. A treatment plan is generated comprising Antihypertensive therapy with labetalol (200 mg orally twice daily), and Corticosteroids (betamethasone 12 mg intramuscularly, two doses 24 hours apart)

Patient B is then hospitalized for closer monitoring. Magnesium sulfate infusion is initiated to prevent seizures, Antihypertensive therapy with labetalol (200 mg orally twice daily), Corticosteroids (betamethasone 12 mg intramuscularly, two doses 24 hours apart) administered to accelerate fetal lung maturity. The system now is configured to acquire Vital signs and fetal status every 6 hours, and reassessed biomarker levels every 24 hours.

Upon discharge, Patient B is provided with a connected pill dispenser to manage her antihypertensive medications and aspirin regimen. The system manipulates the dispenser, tracks compliance and sends reminders.

At 34 weeks, Patient B experiences worsening symptoms, including severe headaches and further elevated blood pressure (160/110 mmHg). The system's predictive model calculates a high probability of imminent adverse outcomes and recommends immediate delivery, as well as a delivery plan: A cesarean section is scheduled within 24 hours. Post-Delivery Monitoring is also applied to Patient B which comprises monitoring Maternal biomarkers and blood pressure monitored daily as well as Neonatal outcomes, including Apgar score and birth weight.

FIG. 2 schematically depicts a layer-like representation of an implementation of the method according to the embodiments of the present invention. The method is a computer-implemented method. The method is carried out by the system 1000. In simple terms, the layer-like representation comprises 3 layers L1, L2 and L3. Layer L1 may also be referred to as input layer, L2 may be referred to as model layer, modeling layer, processing layer and/or analyzing layer. L3 may be referred to as output layer and/or outcome layer.

The input layer L1 may receive a plurality of input 210, 220, 230, which may, inter alia but not limited to, comprise biomarkers such as parental, fetal and/or neonatal biomarkers; a plurality of clinical parameters such as parental, fetal and/or neonatal clinical parameters; demographics, lifestyles and psychometric scores related to the subject and/or a group of subjects; a plurality of environmental parameters; drug treatments such as current drug treatments on the subject and/or recommended drug treatments in guidelines in effect or in force; dosing regimens, drug history related to the subject or a group of subjects; cardiography data (CTG); electroencephalogram data (EEG); electrocardiogram data (ECG), pulse and/or oxygen measurements; data provided, for instance, by sono and variants such as doppler, duplex; magnetic resonance imaging (MRI); X-ray.

These inputs may be processed within the modelling layer L2, wherein a plurality of computer-implemented dynamic model 310, 320, 330 may be applied to the input data to generate processed data, which can be further analyzed and interpreted to generate at least one finding which can be expressed by means of a computer-implementing prediction step as at least one hypothesis as regards the at least one health status of the subject or a group of subjects. The multi-layer computer-implemented method may further make use of the output layer L3, wherein an interpreted data may be provided to a user, such as a physician. Such outputs may, inter alia, comprise PE-related predictions, an evaluation 410, a risk assessment 420 of the PE or any other medical condition of the subject or group of subjects, which may comprise for instance a mother-related risk assessment S1, a fetal-related risk assessment S2 and/or a neonatal-related risk assessment S3. Moreover, the output layer L3 may also comprise one or more therapeutics 430 such as a therapeutic protocol and/or therapeutic approach suggestions as well as the optimization of current and/or future therapeutics.

This is particularly advantageous, as the multi-layer computer-implemented method provide by means of the system 1000 at least one hypothesis as regards the at least one health status of the subject or a group of subjects, wherein the hypothesis is based on discrete available data which can comprise current data and/or historical data. That is, the computer-implemented method can by means of the system 1000 process, analyze and interpret data related, for instance, to PE during pregnancy and its effect on a gestational evolution of a fetus as depicted in FIG. 4, which depicts evolution for healthy pregnant woman 100A and a pregnant woman with PE 100B.

FIG. 3 provides a schematic of a computing device 100. The computing device 100 may comprise a computing unit 35, a first data storage unit 30A, a second data storage unit 30B and a third data storage unit 30C.

The computing device 100 can be a single computing device or an assembly of computing devices. The computing device 100 can be locally arranged or remotely, such as a cloud solution.

On the different data storage units 30 the different data can be stored. Additional data storages can be also provided and/or the ones mentioned before can be combined at least in part.

The computing unit 35 can access the first data storage unit 30A, the second data storage unit 30B and the third data storage unit 30C through the internal communication channel 160, which can comprise a bus connection 160.

The computing unit 30 may be a single processor or a plurality of processors, and may be, but not limited to, a CPU (central processing unit), GPU (graphical processing unit), DSP (digital signal processor), APU (accelerator processing unit), ASIC (application-specific integrated circuit), ASIP (application-specific instruction-set processor) or FPGA (field programable gate array). The first data storage unit 30A may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

The second data storage unit 30B may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

The third data storage unit 30C may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

It should be understood that generally, the first data storage unit 30A (also referred to as encryption key storage unit 30A), the second data storage unit 30B (also referred to as data share storage unit 30B), and the third data storage unit 30C (also referred to as decryption key storage unit 30C) can also be part of the same memory. That is, only one general data storage unit 30 per device may be provided, which may be configured to store the respective encryption key (such that the section of the data storage unit 30 storing the encryption key may be the encryption key storage unit 30A), the respective data element share (such that the section of the data storage unit 30 storing the data element share may be the data share storage unit 30B), and the respective decryption key (such that the section of the data storage unit 30 storing the decryption key may be the decryption key storage unit 30A).

In some embodiments, the third data storage unit 30C can be a secure memory device 30C, such as, a self-encrypted memory, hardware-based full disk encryption memory and the like which can automatically encrypt all of the stored data. The data can be decrypted from the memory component only upon successful authentication of the party requiring to access the third data storage unit 30C, wherein the party can be a user, computing device, processing unit and the like. In some embodiments, the third data storage unit 30C can only be connected to the computing unit 35 and the computing unit 35 can be configured to never output the data received from the third data storage unit 30C. This can ensure a secure storing and handling of the encryption key (i.e., a private key) stored in the third data storage unit 30C.

In some embodiments, the second data storage unit 30B may not be provided but instead the computing device 100 can be configured to receive a corresponding encrypted share from the database 60. In some embodiments, the computing device 100 may comprise the second data storage unit 30B and can be configured to receive a corresponding encrypted share from the database 60.

The computing device 100 may comprise a further memory component 140 which may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM). The memory component 140 may also be connected with the other components of the computing device 100 (such as the computing component 35) through the internal communication channel 160.

Further the computing device 100 may comprise an external communication component 130. The external communication component 130 can be configured to facilitate sending and/or receiving data to/from an external device (e.g., a backup device, a recovery device, a database). The external communication component 130 may comprise an antenna (e.g., Wi-Fi antenna, NFC antenna, 2G/3G/4G/5G antenna and the like), USB port/plug, LAN port/plug, contact pads offering electrical connectivity and the like. The external communication component 130 can send and/or receive data based on a communication protocol which can comprise instructions for sending and/or receiving data. Said instructions can be stored in the memory component 140 and can be executed by the computing unit 35 and/or external communication component 130. The external communication component 130 can be connected to the internal communication channel 160. Thus, data received by the external communication component 130 can be provided to the memory component 140, computing unit 35, first data storage unit 30A and/or second data storage unit 30B and/or third data storage unit 30C. Similarly, data stored on the memory component 140, first data storage unit 30A and/or second data storage unit 30B and/or third data storage unit 30C and/or data generated by the computing unit 35 can be provided to the external communication component 130 for being transmitted to an external device.

In addition, the computing device 100 may comprise an input user interface 110 which can allow the user of the computing device 100 to provide at least one input (e.g., instruction) to the computing device 100. For example, the input user interface 110 may comprise a button, keyboard, trackpad, mouse, touchscreen, joystick and the like.

Additionally, still, the computing device 100 may comprise an output user interface 120 which can allow the computing device 100 to provide indications to the user. For example, the output user interface 110 may be a LED, a display, a speaker and the like.

The output and the input user interface 100 may also be connected through the internal communication component 160 with the internal component of the device 100.

The processor may be singular or plural, and may be, but not limited to, a CPU, GPU, DSP, APU, or FPGA. The memory may be singular or plural, and may be, but not limited to, a volatile or non-volatile memory, such as SDRAM, DRAM, SRAM, Flash Memory, MRAM, F-RAM, or P-RAM.

The data processing device can comprise means of data processing, such as, processor units, hardware accelerators and/or microcontrollers. The data processing device 20 can comprise memory components, such as, main memory (e.g., RAM), cache memory (e.g., SRAM) and/or secondary memory (e.g., HDD, SDD). The data processing device can comprise busses configured to facilitate data exchange between components of the data processing device, such as, the communication between the memory components and the processing components. The data processing device can comprise network interface cards that can be configured to connect the data processing device to a network, such as, to the Internet. The data processing device can comprise user interfaces, such as:
(1) output user interface, such as:
   screens or monitors configured to display visual data (e.g., displaying graphical user interfaces of the questionnaire to the user),
   speakers configured to communicate audio data (e.g., playing audio data to the user),
(2) input user interface, such as:
   camera configured to capture visual data (e.g., capturing images and/or videos of the user),
   microphone configured to capture audio data (e.g., recording audio from the user),
   keyboard configured to allow the insertion of text and/or other keyboard commands (e.g., allowing the user to enter text data and/or other keyboard commands by having the user type on the keyboard) and/or trackpad, mouse, touchscreen, joystick—configured to facilitate the navigation through different graphical user interfaces of the questionnaire.

The data processing device can be a processing unit configured to carry out instructions of a program. The data processing device can be a system-on-chip comprising processing units, memory components and busses. The data processing device can be a personal computer, a laptop, a pocket computer, a smartphone, a tablet computer. The data processing device can be a server, either local and/or remote. The data processing device can be a processing unit or a system-on-chip that can be interfaced with a personal computer, a laptop, a pocket computer, a smartphone, a tablet computer and/or user interface (such as the upper-mentioned user interfaces).

FIG. 5 depicts a schematized example of the use of system 1000. The input to the system may originate from, for example, ultrasound imaging data 205 previously taken in a medical facility, Blood Oxygen Saturation Monitor (SpO2) 215 which may be currently be taken by the subject at home, a sheet of paper 225 with information regarding the subject scanned by the system 1000, and/or an already present database 235 comprising data relating to the subject. System 1000 would be configured to synchronize all these different data types and generate outputs depending on the data itself and the time relevance of these inputs with respect to the type of input they represent. Storing component 1400 may comprise a server and allow transfer of data from one device to another. The outputs of system 1000, which may comprise outputs of the modeling layer, may be displayed to the subject directly, may be sent out to another end-user device 440, which may belong to a medical professional, and/or may operate an external medical device such as a portable pill dispenser 450 in the subject's possession to control the medicine intake of the subject.

It is noted that not all the drawings carry all the reference signs. Instead, in some of the drawings, some of the reference signs have been omitted for sake of brevity and simplicity of illustration. Embodiments of the present invention will now be described with reference to the accompanying drawings.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of: a first option and a second option” is intended to mean the first option or the second option or the first option and the second option.

While in the above, a preferred embodiment has been described with reference to the accompanying drawings, the skilled person will understand that this embodiment was provided for illustrative purpose only and should by no means be construed to limit the scope of the present invention, which is defined by the claims.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), (Y2), . . . , followed by step (Z). Corresponding considerations apply when terms like “after” or “before” are used.

1. A method for continuously predicting the health status of a subject and/or an ambulatory subject, the method comprising
receiving, by at least one processing component, at least one subject-related dynamic property data,
receiving, by the at least one processing component, at least one subject-related covariate,
processing, by the at least one processing component, the at least one subject-related dynamic property data and the at least one subject-related covariate data to generate a subject-related processed dataset,
generating, by at least one analyzing component at least one health status hypothesis based on the subject-related processed dataset, and
predicting, by at least one computing component at least one health status based on the at least one health status hypothesis based on a computer-implemented dynamic model, wherein the at least one health status comprises preeclampsia and/or fetal growth-related conditions.

2. The method according to claim 1, wherein the at least one health status hypothesis comprises a correlation to at least one medical condition of the subject, wherein the method further comprises predicting a dynamic behavior of the at least one health status of the subject.

3. The method according to claim 1, wherein the at least one subject-related covariate comprises at least one of
at least one biomarker, wherein the at least one biomarker is related to at least one medical condition, wherein the at least one biomarker comprises at least one of: soluble Fms-like tyrosine kinase (sFlt-1); placental growth factor (PIGF); neurofilament (NfL); C-terminal portion of arginine vasopressin (Copeptin); Albumin; Liver transaminase; Urea; Hemoglobin; Thrombocytes; Creatinine; Albuminuria; Proteinuria; estimated glomerular filtration rate (eGFR); creatinine clearance (CrCl); at least one additional kidney function measure; placental biomarkers comprising at least one of placental RNAs, and placental proteins; endothelial/cardiovascular biomarkers comprising at least one of endothelial RNAs, and endothelial proteins; or any combination thereof;
at least one mother-related covariate, comprising at least one of: age; weight;
height; body mass index (BMI); gravidity; parity; number of fetuses in a current pregnancy;
ethnicity; body temperature; heart rate; heart rate variability; respiration rate; early membrane rupture; leukocytes; history of preeclampsia (family and mother), comorbidities comprising at least one of gestational diabetes, obesity, cardiovascular/renal/kidney/thyroid diseases, autoimmune conditions, anemia, antiphospholipid syndrome, sexually transmitted diseases, and headache; smoking habits before and/or during pregnancy; blood oxygen saturation (SpO2); blood pressure (systolic/diastolic); utero-placental perfusion parameters; doppler measurements of: umbilical artery, middle cerebral artery, cerebroplacental ratio, uterine artery, fetal descending aorta, ductus venosus, umbilical vein, inferior vena cava, pulsatility index in uterine arteries; soft-tissue parameters comprising at least one of fractional arm volume and fractional thigh volume; and at least one measurement of the at least one biomarker;
at least one fetus-related covariate comprising at least one of: gender; fetal weight during pregnancy; fetal biometric parameters comprising at least one of femur length, abdominal circumference, head circumference, mid-thigh circumference and biparietal diameter; rates of small for gestational age; gestational age; heart rate, heart rate variability; respiration rate; utero-placental perfusion parameters; and at least one measurement of the at least one biomarker;
at least one neonate-related covariate, comprising at least one of: gender: birth weight; body weight; body length; gestational age at birth; postnatal age; temperature; heart rate; heart rate variability; respiration rate; breastfeeding; exclusive breastfeeding duration; pH value; breath aid; oxygen demand; blood oxygen saturation (SpO2); blood pressure (systolic/diastolic); Apgar scores; at least additional neonatal biometric parameters; and at least one measurement of the at least one biomarker; and
at least one environmental covariate comprising at least one of: country of residence, country of birth, day and time of birth, humidity conditions at birth, and surrounding temperature at birth.

4. The method according to claim 1, wherein the method comprises
generating at least one threshold, wherein the at least one threshold expresses an indication of at least one potential medical condition;
outputting at least one potential medical condition, wherein the at least one potential medical condition comprises at least one of: seizures; respiratory; cardiovascular; hematological dysfunction; endocrine; renal; hepatic; uteroplacental dysfunction; fetal growth restriction; unplanned preterm birth; placental abruption; hemolysis elevated liver enzymes, low platelets (HELLP) syndrome; and eclampsia;
determining a minimum threshold value for the at least one threshold, and
determining a maximum threshold value for the at least one threshold,
determining a baseline for the at least one subject-related data, and
determining comprises at least one intermediate threshold value, wherein the at least one intermediate threshold value comprises at least one value between the minimum threshold value and the maximum threshold value.

5. The method according to the claim 4, wherein the method comprises
correlating at least a range of each of the at least one intermediate threshold value to the at least one medical condition,
generating an interpreted dataset based on the correlation step, and
outputting an automated report indicating the at least one potential medical condition.

6. The method according to claim 1, wherein the method comprises
determining at least one medical condition change indicator,
monitoring changes of the at least one medical condition change indicator,
generating at least one medical condition change indicator trend, and
predicting an evolution of at least one of the at least one medical condition, wherein the prediction is based on the at least one medical condition change indicator trend,
wherein the method comprises monitoring at least one value change of the at least one subject-related property, the method comprising
recording an initial value of the at least one subject-related property,
recording at least one subsequent value of the at least one subject-related property,
contrasting the initial value with at least one of the at least one subsequent value,
generating a compared value data, and
outputting a subject-related property hypothesis based on the compared value data, wherein the step of recording at least one subsequent value comprises recording one current value of the at least one subject-related property, wherein the current value is different from the initial value.

7. The method according to claim 2, wherein the method comprises
feeding data to the at least one server,
training the computer-implemented dynamic model based on data fed to the at least one server, and
generating an adjusting function based on the training data, wherein the adjusting function is suitable for adjusting any steps of the method according to any of the preceding claims,
triggering at least one action suggestion based on the at least one health status hypothesis,
displaying the at least one action suggestion to a user, and
prompting the user to input at least one of: acceptation of at least one of the at least one action suggestion, and rejection of at least one of the at least one action suggestion.

8. The method according to claim 1, wherein the method comprises
determining at least one drug based on the at least one health status, wherein the at least one drug is suitable for at least one of
preventing occurrence of the at least one medical condition and/or the at least one health status, and
treating the at least one medical condition and/or the at least one health status;
generating at least one dosing regimen of the at least one drug;
optimizing the at least one dosing regimen, wherein the at least one dosing regimen comprises at least one of: the at least one drug, the at least one drug administration route, at least one dosing regimen, at least one drug administration duration, and at least one drug administration frequency, wherein the step of optimizing the at least one dosing regimen is based on the at least one health status hypothesis.

9. A system for continuously predicting the health status of a subject and/or an ambulatory subject, the system comprising
at least one processing component configured to
receive at least one subject-related dynamic property data,
receive at least one subject-related covariate, and
process the at least one subject-related dynamic property data and the at least one subject-related covariate data to generate a subject-related processed dataset,
at least one analyzing component configured to
analyze the subject-related processed dataset, and
generate at least one health status hypothesis based on the subject-related processed dataset, and at least one computing component configured to
predict at least one health status based on the at least one health status hypothesis, and perform the method according to claim 1, wherein the at least one health status comprises preeclampsia and/or fetal growth-related conditions.

10. The system according to claim 9, wherein the system comprises
at least one storing component configured to store data relevant to the at least one health status of the subject, wherein the at least one health status hypothesis comprises a correlation to at least one medical condition of the subject,
wherein the system is configured to predict a dynamic behavior of the at least one health status of the subject.

11. The system according to claim 9, wherein the system is configured to
generate at least one threshold, wherein the at least one threshold expresses an indication of at least one potential medical condition; and
output at least one potential medical condition, wherein the at least one potential medical condition comprises at least one of: seizures; respiratory; cardiovascular; hematological dysfunction; endocrine; renal; hepatic; uteroplacental dysfunction; fetal-growth restriction; unplanned preterm birth; placental abruption; hemolysis elevated liver enzymes, low platelets (HELLP) syndrome; and eclampsia.

12. The system according to claim 11, wherein the at least one analyzing component is configured to
determine a minimum threshold value for the at least one threshold,
determine a maximum threshold value for the at least one threshold,
determine at least one intermediate threshold value,
correlate at least a range of each of the at least one intermediate threshold value to at least one medical condition,
generate an interpreted dataset based on the correlation step,
output an automated report indicating at least one potential medical condition,
determine at least one medical condition change indicator,
monitor changes of the at least one medical condition change indicator,
generate at least one medical condition change indicator trend, and
predict an evolution of at least one of the at least one medical condition based on the at least one medical condition change indicator trend.

13. The system according to claim 9, wherein the system comprises at least one monitoring component configured to monitor at least one value change of the at least one subject-related property, wherein the at least one monitoring component is further configured to
record an initial value of the at least one subject-related property,
record at least one subsequent value of the at least one subject-related property,
contrast the initial value with at least one of the at least one subsequent value,
generate a compared value data,
output a subject-related property hypothesis based on the compared value data,
record one current value of the at least one subject-related property, wherein the current value is different from the initial value.

14. The system according to claim 10, wherein the system is configured to
feed data to the at least one server, and
train the computer-implemented dynamic model based on data fed to the at least one server, and
generate an adjusting function based on the training data, wherein the adjusting function is suitable for adjusting any configuration of the system according to any of the preceding claims;
trigger at least one action suggestion based on the at least one health status hypothesis, wherein the system is configured to display the at least one action suggestion to a user and to prompt the user to input at least one of: acceptation of at least one of the at least one action suggestion, and rejection of at least one of the at least one action suggestion.

15. The system according to claim 9, wherein the system comprises at least one imaging component, comprising at least one X-ray, a Magnetic Resonance Imaging, and ultrasound device, configured to at least one of
capture at least one image data of the subject; and
send the at least one image data of the subject to the at least one processing component,
wherein the at least one image data comprises data relevant to at least one medical condition and/or at least one potential medical condition of the subject.

16. The method according to claim 1, further comprising designing a treatment protocol based on the at least one health status hypothesis, wherein the treatment protocol comprises at least one treatment drug and a treatment regimen.

17. The method according to claim 1, further comprising diagnosing a medical condition of a subject based on the at least one health status hypothesis by generating at least one diagnostic finding comprising at least one medical condition of the subject.
